SEQUENCE LISTING (154 sequences)

SEQ ID NO: 1 - DNA (Genomic DNA), 4047 bp; double-stranded, linear
aagctttttgctttccttccccgggaaaggccggggccagagacccgcactcggaccaggcgggggctgcggggccagagtgggctggggagggctgggagggcgtctggggccggctcctccaggctgggggccgccagctccgggaaggcagtcctggcctgcggatggggccgcgcgtggggcccggcggggcggcctcgggaggcgtccaggctgcgggagcgggaggagcggccgtgcgggcgccagcgccgtgggtggaggtcgccgtccctcctgaggggcagccagtgcgtttgggacccgggagcagagcccgcgcctccccagcggcctccccgggggtctcaccgggtcacccgagagcggaggccccggctccgcagaaacccggggcggccgcggggaagcagcgccctcaggcgtcggaggagcccccagaaggacctcgcgccttcccgccgggctccgaccgcctgggttcggtgcgggacggcccaggccgccaggacccccaagcgcagctcagtctgcggggcacgacccagaggccagcagcagaggacggggccggggccgggagagggcggggagggcgctcctgggaggtcaaggccagggctagactttcagggtcatggcctggcccctcatccccagggaggtgagggggctctgtgagcagagggggccccggtggagaaggcgctgctagccaggggcggggcaggagcccaggtggggacttaagggtggctgaagggaccctcaggctgcagggatagggagggaagctaggggtgtggcttggggaggtgctgggggaccgcgggcgccctttattctgaagccgaatgtgctgccggagtccccagtgacctagaaatccatttcaagattttcaggagtttcaggtggagacaaaggccaggcccaggtgaaaatgtggcagtgacagagtatggggtgagaaccacggagagaggaagtccccgaggcggatgatgggacagagagcggggaccagaattttttaaaacgcatctgagatgcgtttggcagactcatagttgttttcctttcacggagaaagtgtgggcagaagccagctctaaagcccaggctgcccagcctgcactggcagagctgacggaaggccagggcagagccttccctccctgtcacagacatgagccctggagatctggaatgaggcagatgtgcccagggaaagctgatccgccccgacccagggccccccgggtgcccctttgagcgtggaatcgttgccaggtcatggctccctgctatcgaacaccggacacgggtcgtgtgctgcacctggcagttgcaggaccgacacccacaatgccttaagaggtgatgactgccttccaggggcctggctggctgacactttgcatggctcctggagaagagggattgagtggagtccacgggtcatggccacgtcctgggtgctgcctctgaggcagggcccggctggggtgagaaggggctggagacaggttcctgccagttcagcctctaaccggtggtcttcatgcctaggaacccactgggggcttatgaaactgcaggtggctgagtccttgccatggggtctctccttcaggaggtctgggtggggccggagactgtaccccacaaagggtcccaggtgaggcggatgtggcctggcgctgtgtggctctggacctagtccttgggcttgggctggcgcccagggcctgggcttgagacagctgtgacgcaggcaagccatttaccccgtttgtggggacattacatcttcctagcttggaacacacaggcagccagggttgttatccacattcctcctccatgttcttctcttgagaacttttaccaggtatgtcaggagctgggctccaccagggagactcaagtggaaagccctcatccttgtcctccaggagacaggaaaacctatggttacaattccagggacaagagcgatgcatgtgaggtgtggcaaatctcactgttcaactggagaaatcagagacagcttcctggaggcagtgacacctggacaggcttctccacaggaggaagcgagtgagagaagccaactgggatggacccatcatgtagggggaacagtgcgcgcagaaccaacaaccacccccaccctaggcccagagctcacggagagagctgggcctctcggggtgactacatagttccctgctggatcttaggtcttgtccttgggcagctctgctgagacctctatgcctgttccaggctgcaccaaggttttgtgactattggtctggggttgttttgcagcaactgaagtgttctgttgtaaaacaggcacttgatttgctggaaggaatgctgtttgttcttgctgcgacaaacattgagcagcatttagtgggcggtttatatcttgtggagtaatgggtgtttttgaagtctgtcctgggtactgcacattaaaaggaatatcattttctgaaacattgctattttccacaccagaaatcatatcctcttgctggtccatgtctgaagaccttacacgagaaagtcttaatgtaagtttagtagagtccttggatggagaactaattatatcatacattgccgctttctcactctgctctttttcatccttgcctaatttcattttcttctgcttcttttgttttctttctggagaatctagcaagatatctggtggaacatctcgaggtgatgaacaaggtagagactgagattgtaggattaaaggtggtcttgagcctttaggagttccttcacttccagcaggggagcatactggctgtggagatctcaagggaaaagatgcagcattcctcattgttgaagaatctccatcgtcactacttagcctgtgcaccatgtgtaggtagtcctcacttgaaccatgtctaggattatcagcatgatgattagctgaattgccagacaacggaccagaaactttattatcatgtatgtttctcaaaccacctgcaacaatgggacttgataccgatgcttgttgcatctgtggatgtgttgtgtaacttgaaggatgggaatatggcatgtatcctgcagggctttgtggggcgtatggactaggcactgggctattttgctgtggcataaatctgttcccagagcttgtctgtggtggcacaaaccggctggaggggctatgtgagatagtggtttgttgataattggaagatgcaggactactgtgcatggaattctgagaaagtttatactgagacatcatcattccactttgtacatatctgttctgcatgcttttctccctgaaaacattaggactccttgccaggacggcctgcaacaagactggtatgtcaccttctgggtcatcactgccaaggttatctttcaactctatgtgatctgttgatacctggttgaggctatggacaagctgtgaaaccaaattgtcatccctacaagccaaaaggcagttcacctcttctgctattcgtgcattaaagagaaggctctttgtagttgtagcaggtaaaggagatggaagaggcagctggttcaggaggtctgtgagactagcaatccccgcaagagtagtaatggggacatggggcatatccccattcatcctgaatttctggaatggtgttgcctataaaagtacttagttcaggtgccagctgtcattacttcccatttcccaaacactgggcgaatcggcgtctgaatccaaggggaggccgaggccgctgtggcgagagactataatccgggccgggagggggggcggctacggctcctcttccgtctcctcagtgcggggaacatgtagagccggggggagaccagccgagaagacaaatcgttgcttcttcttcctcctcctcctccttctcccacatagaaacactcacaaacacccgaccacgggcccgagctaccgggggggcatcgccgcgggcccgggaaccaattctcctgtcggcgggggcgtcctttggatcc

SEQ ID NO: 2 - DNA (Genomic DNA), 739 bp; double-stranded, linear
ggatccaaaggtcaaactccccacctggcactgtccccggagcgggtcgcgcccggccggcgcgcggccgggcgcttggcgccagaagcgagagcccctcggggctcgcccccccgcctcaccgggtcagtgaaaaaacgatcagagtagtggtatttcaccggcggcccgcagggccggcggaccccgccccgggcccctcgcggggacaccgggggggcgccgggggcctcccacttattctacacctctcatgtctcttcaccgtgccagactagagtcaagctcaacagggtcttctttccccgctgattccgccaagcccgttcccttggctgtggtttcgctggatagtaggtagggacagtgggaatctcgttcatccattcatgcgcgtcactaattagatgacgaggcatttggctaccttaagagagtcatagttactcccgccgtttacccgcgcttcattgaatttcttcactttgacattcagagcactgggcagaaatcacatcgcgtcaacacccgccgcgggccttcgcgatgctttgttttaattaaacagtcggattcccctggtccgcaccagttctaagtcggctgctaggcgccggccgaagcgaggcgccgcgcggaaccgcggcccccggggcggacccgcgggggggaccgggccgcggcccctccgccgcctgccgccgccgccgccgccgcgcgccgaagaagaagggggaaa

SEQ ID NO: 3 - DNA (Genomic DNA), 233 bp; double-stranded, linear
caagagtggcggccgcagcaggccccccgggtgcccgggcccccctcgagggggacagtgcccccgccgcgggggccccgcggcgggccgccgccggcccctgccgccccgacccttctccccccgccgccgcccccacgcggcgctcccccggggaggggggaggacggggagcgggggagagagagagagagagagggcgcggggtggctcgtgccgaattcaaaaagctt

SEQ ID NO: 4 - DNA (Genomic DNA), 2998 bp; double-stranded, linear
ggatccaaagaattcggcacgaggtagtcacggctcttgtcattgttgtacttgacgttgaggctggtgagcttggaaaagtcgatgcgcagcgtgcagcaggcgttgtagatgttctgcccgtccagcgacagcttggcgtgctgggcgctcacggggtccgcatactgcagcagggcctggaactggttgttcttggtgaaggtgatgatcttcaacactgtgccgaacttggagaaaatctggtgcagcacatccagggtcacagggtagaagaggttctccacgatgatcctgagcacggggctctgcccggccatcgccatccctgcatccacggccgccgccgaggcagccaaggccaggttccccgactggaccgagttcaccgcctgcagggccgcctgggcccgcgcctggttgggagagctgtcggtcttcagctccttgtggttggagaactggatgtagatgggctggccgcgcagcacaggggtcaccgaggtgtagtagttcaccatggtattggcagcctcctccgtgttcatctcgatgaaggcctggtttttccccttcagcatcaggaggttggtgaccttcccaaagggcagccccagggagatgacttccccctccgtgacgtcgatggggagcttccggatgtggatcactctagaggggacgcctgcacttcggctgtcacctttgaacttcttgctgtcatttccgtttgctgcagaagccgagttgctgctcatgataaacggtccgttagtgacacaagtagagaaaagctcgtcagatccccgctttgtaccaacggctatatctgggacaatgccgtccatggcacacagagcagacccgcgggggacggagtggaggcgccggaatcctggagctagagctgcagattgagttgctgcgtgagacgaagcgcaagtatgagagtgtcctgcagctgggccgggcactgacagcccacctctacagcctgctgcagacccagcatgcactgggtgatgcctttgctgacctcagccagaagtccccagagcttcaggaggaatttggctacaatgcagagacacagaaactactatgcaagaatggggaaacgctgctaggagccgtgaacttctttgtctctagcatcaacacattggtcaccaagaccatggaagacacgctcatgactgtgaaacagtatgaggctgccaggctggaatatgatgcctaccgaacagacttagaggagctgagtctaggcccccgggatgcagggacacgtggtcgacttgagagtgcccaggccactttccaggcccatcgggacaagtatgagaagctgcggggagatgtggccatcaagctcaagttcctggaagaaaacaagatcaaggtgatgcacaagcagctgctgctcttccacaatgctgtgtccgcctactttgctgggaaccagaaacagctggagcagaccctgcagcagttcaacatcaagctgcggcctccaggagctgagaaaccctcctggctagaggagcagtgagctgctcccagcccaacttggctatcaagaaagacattgggaagggcagccccagggtgtgggagattggacatggtacatcctttgtcacttgccctctggcttgggctcctttttctggctggggcctgacaccagttttgcccacattgctatggtgggaagagggcctggaggcccagaagttgctgccctgtctatcttcctggccacagggcttcattcccagatcttttccttccacttcacagccaacggctatgacaaaaccactccctggccaatggcatcactcttcaggctggggtgtgctccctgaccaatgacagagcctgaaaatgccctgtcagccaatggcagctcttctcggactcccctgggccaatgatgttgcgtctaataccctttgtctctcctctatgcgtgcccattgcagagaaggggactgggaccaaaggggtggggataatggggagccccattgctggccttgcatctgaataggcctaccctcaccatttattcactaatacattttatttgtgttctctaatttaaaattaccttttcatcttgcttgattttccttcagctaaattagaaatttgtagtttttcccctaaaaaattcaatggcattctttcttataaattacattctctgattttcttgtcagcctgcttcaaggaaatccatgtgttcaaaatgcttgctcgcagtttgctccataccaaatggttgcttaacccaaatatctgagcagcaaattgagctgatccttctggagaaagtacggttgaacagccaagaccactgggtagtcgaagagaagaccacacatcctgaactccccagtctggtgtgaggggaggacagctgataactggatatgcagtgttcccagacatcactggtcccaaaccattacttctgcctgccactgccacaaatacagtaggaatgccatccccttcatactcagctttaatcctcagagtttcatctggtcctttatgcgcagatgttactcgaagttcacatggaatgccaaaatttccacaggccttcttgattttttcacagtgaccaagatcagaagtagagcccatcaacactacaaccctgcactgactttctgatttcaaaagcaactctactctctctgcaacccactcaaagtttttctttaccatttggagcccttcaggagttacttctttgaggtcccgataagactgtttgtctttctgttggcttcgatctcctgatggccagagtctccaggaatcattgtcaataacatcagcaagaacaatttctttggtggttacatcaacaccaaattcaatcttcatatcaaccagtgtacaattctggggcaaccaggatttctccagtatttcaaatatagcctgtgtagcatctcgtgccgaattcaaaaagctt

SEQ ID NO: 5 - DNA (Genomic DNA), 4152 bp; double-stranded, linear
aagctttttgtgaaaaccctaggatatgtcccctccctcaccacacccaaccccccgcccctgccccaggacatgacgatgcctcacacacacacacacacacacatacacacaaggccgtgagctgcacgcaggaacatgggctgcactcacgacaacattgaaaaaatatacattatatatgtacacccggggcccccacgtcccctcccgtccccgcagcctggccacaccaggtcacggaggaggggccggggctgcaggacctcaggactgcaagggcaggaagggaaacaggacaagaaaggaaggaagttggaaaggagggagaaatggggtccccagactgaaatggaaatgaggtggggcgatcataagagaagcagggacgatggtccagctgagggagccctgcagagggggaaaagcttcccatggacaggagagagaagggaaggggagaggagagggtttccttcaatcccacccccagccccagccccagccccagccattgcaatcgtcaccctctccccaacacagtgagtgctaagggggcagctgccattgggggtagaaaggcagctgaagtccagcccactttccaacccagccagccccagtgcaaggggcacaccaggagcatgacagcccagaagtgagggatggggggccgggggaggggcagggcggactccagagggcccgctggggttttgaaatgaaaggaggactggttctgaagcctctctccctcttggtctctgtgttcccagaaagtccttctcccatgtctggagtgtctgtttcaccagggcagaattccccctctgcgtggggagaggtgtaggccttagtagcggtgtgggggggtctcgatgatgcgtctctcgtcgctgctgggggaatcggccacctccgagtcactgctgtcctcatcctcctgctggcccccaacagcccccgtcacacaggactgccgattctggtaggactccatggggttcacaatgatggtgagagctgagtcatcccagaagaggtctgggtccttggggtcactggaggcccctggaggcccgccggcccctgagacgcggcggtgaagggaatggatgcgcaccaggcccaggacgaccatgagcaccaggaagcccacgcacaccacaatgatgagggttgcggcgctgggtatcatggagtttctgtgggagctggctaggctgtgtccagccatctcaggcgggggctggtgaccacggtgcaggaactgctgggagctgagcacgtggctggggtgggcaacccggttcatgctgtgcaggacattgacctccacgatgaattcattgctggagtaacggccattcatttccgagcaggaaagccggaacttcctggtgtagagggcagctccgtgtcgcagccgataacgagcctgcctcaggatctcttcatacacagtgatgctctccaccccagcaatagtgaggtaggcagatgtgttggtgagctccagcccccgctgctgcagagaggttgtgtccaggagcaggctttcccgctcgggatccaggtcatcccccaccagagaaatttcacagccatccaggttgtgcacaatctcatccgacatgcgtgtgtctgtcactgtgccctgccaactctcatcctttttggcctccacctggtgagaaatggagcaggtgatttgaagatcagggaacaaagggacgccgttggttccctcaaagtccacagctgggcgggcaaaatgagcagtgccactcagcaggatctggggggcgtcaggctgaaggacgaccacgtagccctccacttcagggatggagacgcaggactcttcgctgaagcacttgacagcagtggtgaggcgcaggggcctgacgccgggcgtggcaaagcgcagagtgttcatgtaagccacatgctgcagggcatggttgaaggtctccacatcatccccctccagggtgagcagggactgtgaggggttcacgtggaccttcatgcctttgcccaggctctcgaaatccctatagtccagcccctcccgacatgcatagaggcactcgatgacctcgcggctctccaggcgacctgagcgcacgctgaaaccagccaggtagccatggaagtagtggtggatcgacaaagggtctccttgggtggtgtctgtactgttgtctcccttttccttctctttgttcttctcctcagtccagcaggccccaatcatgagagcaggctcccttcggggtgggtggatgaggccattgtcatggatgagggcagggtcgaaggagatgccgtcggtatagagtgtgactgtggggaactcgaggttcagagcgtagtggtgccactcatcatcacagacctgctccagcttccagaggaacttgactgggcgggcactctcaagcaggggccagtagaggaaggcaatcctacagccgtggacagtcagcgagtagtgagagaagccgtcctcattctggacagtgttacatacgatggtttcctcttccttcttgcccttgttgggagttacgccatgcttcatccagaaggacagggtgaagtggtcactgaggctgtcctggggcccagagcccagcccactggggccacccaggggcacctgcacagcctgggtgccattgaaccagtagatcaggctgctgtcctggctgtagtgcaccgagagtcctgctgtccagttggcattggggccaggcatgggcaacagatccacttccccagtggcagcaccacagagtttccgcagcgcccgctctgagtagttgtcacggtcacagcccttggccacatggctggtctgcagctctatggtggcctgaatgttccagagtggttcatcacaggtctccaggcggataccagggaacaaagccaagctcccagcacctggtgcatattcgatccttttgttccagccttgccagctgggtttacaggtgggcttcacctgaatctccacctcagcatcatctgctgcccgcttcttcccacagtcataagctgtcactgtaaacttatagagcctctcaccactgtactgcagcttctctgtgttctcaatgttcccgtcattgtcaatgaggaaaggggtgttgggtgtgagaatctcatagtagcagatctggctgtactggggggagcagtcaccgtcaatggcttccacccgcaggatgcgatcgtacagcttcccctctgtcacagccgcacgatacagccgttccacaaacactggggcaaactcgttcacatcgttgacccgcacatgcacagtggccttgtgggacttcttggtgttggccccgtcggggccctcgccacagtcataggcctggatggtgaaggtgtgttccttctgggcctcgcagtccacaggctccttggcccggatcagcccctctcctgtcgccttgtcaaggatcacagcctcaaagggcaccccagacccatggagccggaagccgcagatctcacctgcatagcgcagcggggcatccttgtccaaggcaaagagtggtggattcagtaggaccgtgttgtcattctccatgacgatgccctggtactctgcctcaatccatggcttgtgcttgttggctttgttacaggagcaggacgcgagcagagaggccagcagaaggggcagcagcaggagggtcatggtgcggcgtggggcagggcagggccaggcgtttgcctcccctgggagcctccagcctgcggattccaccttgcgggagggatacagggggggaaaaccaaaataaaacgtcaaataaattgtgtaggaggagtccagcttaggaccgggccagagccaggccaggctcggggagggggcctctgcaggttcagaggatcactgctgccaccaccgccaccctgggagccagttattttgccatggccttgattgcaacagctgcctcctctgtcatggcagacagcaccgtgatcaggatctcttctccacagtcgtacttctgctcaatctccttgccaaggtctccctcagggagacgaaggtcctctcgtacctccccgctgtcctggagcagtgataggtacccatcctggatctttggatcc

SEQ ID NO: 6 - DNA (Genomic DNA), 3117 bp; double-stranded, linear
ggatccaaagattcggcacgagtggccacatcatgaacctccaggcccagcccaaggctcagaacaagcggaagcgttgcctctttgggggccaggaaccagctcccaaggagcagccccctcccctgcagcccccccagcagtccatcagagtgaaggaggagcagtacctcgggcacgagggtccaggaggggcagtctccacctctcagcctgtggaactgccccctcctagcagcctggccctgctgaactctgtggtgtatgggcctgagcggacctcagcagccatgctgtcccagcaggtggcctcagtaaagtggcccaactctgtgatggctccagggcggggcccggagcgtggaggaggtgggggtgtcagtgacagcagctggcagcagcagccaggccagcctccaccccattcaacatggaactgccacagtctgtccctctacagtgcaaccaaggggagcccgcatcctggagtgggagtcccgacttactataaccaccctgaggcactgaagcgggagaaagcggggggcccacagctggaccgctatgtgcgaccaatgatgccacagaaggtgcagctggaggtagggcggccccaggcacccctgaattctttccacgcagccaagaaacccccaaaccagtcactgcccctgcaacccttccagctggcattcggccaccaggtgaaccggcaggtcttccggcagggcccaccgcccccaaacccggtggctgccttccctccacagaagcagcagcagcagcagcaaccacagcagcagcagcagcagcagcaggcagccctaccccagatgccgctctttgagaacttctattccatgccacagcaaccctcgcagcaaccccaggactttggcctgcagccagctgggccactgggacagtcccacctggctcaccacagcatggcaccctaccccttcccccccaacccagatatgaacccagaactgcgcaaggcccttctgcaggactcagccccgcagccagcgctacctcaggtccagatccccttcccccgccgctcccgccgcctctctaaggagggtatcctgcctcccagcgccctggatggggctggcacccagcctgggcaggaggccactggcaacctgttcctacatcactggcccctgcagcagccgccacctggctccctggggcagccccatcctgaagctctgggattcccgctggagctgagggagtcgcagctactgcctgatggggagagactagcacccaatggccgggagcgagaggctcctgccatgggcagcgaggagggcatgagggcagtgagcacaggggactgtgggcaggtgctacggggcggagtgatccagagcacgcgacggaggcgccgggcatcccaggaggccaatttgctgaccctggcccagaaggctgtggagctggcctcactgcagaatgcaaaggatggcagtggttctgaagagaagcggaaaagtgtattggcctcaactaccaagtgtggggtggagttttctgagccttccttagccaccaagcgagcacgagaagacagtgggatggtacccctcatcatcccagtgtctgtgcctgtgcgaactgtggacccaactgaggcagcccaggctggaggtcttgatgaggacgggaagggtcttgaacagaaccctgctgagcacaagccatcagtcatcgtcacccgcaggcggtccacccgaatccccgggacagatgctcaagctcaggcggaggacatgaatgtcaagttggagggggagccttccgtgcggaaaccaaagcagcggcccaggcccgagcccctcatcatccccaccaaggcgggcactttcatcgcccctcccgtctactccaacatcaccccataccagagccacctgcgctctcccgtgcgcctagctgaccacccctctgagcggagctttgagctacctccctacacgccgccccccatcctcagccctgtgcgggaaggctctggcctctacttcaatgccatcatatcaaccagcaccatccctgcccctcctcccatcacgcctaagagtgcccatcgcacgctgctccggactaacagtgctgaagtaaccccgcctgtcctctctgtgatgggggaggccaccccagtgagcatcgagccacggatcaacgtgggctcccggttccaggcagaaatccccttgatgagggaccgtgccctggcagctgcagatccccacaaggctgacttggtgtggcagccatgggaggacctagagagcagccgggagaagcagaggcaagtggaagacctgctgacagccgcctgctccagcattttccctggtgctggcaccaaccaggagctggccctgcactgtctgcacgaatccagaggagacatcctggaaacgctgaataagctgctgctgaagaagcccctgcggccccacaaccatccgctggcaacttatcactacacaggctctgaccagtggaagatggccgagaggaagctgttcaacaaaggcattgccatctacaagaaggatttcttcctggtgcagaagctgatccagaccaagaccgtggcccagtgcgtggagttctactacacctacaagaagcaggtgaaaatcggccgcaatgggactctaacctttggggatgtggatacgagcgatgagaagtcggcccaggaagaggttgaagtggatattaagacttcccaaaagttcccaagggtgcctcttcccagaagagagtccccaagtgaagagaggctggagcccaagagggaggtgaaggagcccaggaaggagggggaggaggaggtgccagagatccaagagaaggaggagcaggaagaggggcgagagcgcagcaggcgggcagcggcagtcaaagccacgcagacactacaggccaatgagtcggccagtgacatcctcatcctccggagccacgagtccaacgcccctgggtctgccggtggccaggcctcggagaagccaagggaagggacagggaagtcacgaagggcactacctttttcagaaaaaaaaaaaaaaaaacaaaaagctt

SEQ ID NO: 7 - DNA (Genomic DNA), 3306 bp; double-stranded, linear
gaattcggcacgaggtcagtttcctgtggaacacagaggctgcctgtcccattcagacaacgacggatacagaccaggcttgctctataagggatcccaacagtggatttgtgtttaatcttaatccgctaaacagttcgcaaggatataacgtctctggcattgggaagatttttatgtttaatgtctgcggcacaatgcctgtctgtgggaccatcctgggaaaacctgcttctggctgtgaggcagaaacccaaactgaagagctcaagaattggaagccagcaaggccagtcggaattgagaaaagcctccagctgtccacagagggcttcatcactctgacctacaaagggcctctctctgccaaaggtaccgctgatgcttttatcgtccgctttgtttgcaatgatgatgtttactcagggcccctcaaattcctgcatcaagatatcgactctgggcaagggatccgaaacacttactttgagtttgaaaccgcgttggcctgtgttccttctccagtggactgccaagtcaccgacctggctggaaatgagtacgacctgactggcctaagcacagtcaggaaaccttggacggctgttgacacctctgtcgatgggagaaagaggactttctatttgagcgtttgcaatcctctcccttacattcctggatgccagggcagcgcagtggggtcttgcttagtgtcagaaggcaatagctggaatctgggtgtggtgcagatgagtccccaagccgcggcgaatggatctttgagcatcatgtatgtcaacggtgacaagtgtgggaaccagcgcttctccaccaggatcacgtttgagtgtgctcagatatcgggctcaccagcatttcagcttcaggatggttgtgagtacgtgtttatctggagaactgtggaagcctgtcccgttgtcagagtggaaggggacaactgtgaggtgaaagacccaaggcatggcaacttgtatgacctgaagcccctgggcctcaacgacaccatcgtgagcgctggcgaatacacttattacttccgggtctgtgggaagctttcctcagacgtctgccccacaagtgacaagtccaaggtggtctcctcatgtcaggaaaagcgggaaccgcagggatttcacaaagtggcaggtctcctgactcagaagctaacttatgaaaatggcttgttaaaaatgaacttcacggggggggacacttgccataaggtttatcagcgctccacagccatcttcttctactgtgaccgcggcacccagcggccagtatttctaaaggagacttcagattgttcctacttgtttgagtggcgaacgcagtatgcctgcccacctttcgatctgactgaatgttcattcaaagatggggctggcaactccttcgacctctcgtccctgtcaaggtacagtgacaactgggaagccatcactgggacgggggacccggagcactacctcatcaatgtctgcaagtctctggccccgcaggctggcactgagccgtgccctccagaagcagccgcgtgtctgctgggtggctccaagcccgtgaacctcggcagggtaagggacggacctcagtggagagatggcataattgtcctgaaatacgttgatggcgacttatgtccagatgggattcggaaaaagtcaaccaccatccgattcacctgcagcgagagccaagtgaactccaggcccatgttcatcagcgccgtggaggactgtgagtacacctttgcctggcccacagccacagcctgtcccatgaagagcaacgagcatgatgactgccaggtcaccaacccaagcacaggacacctgtttgatctgagctccttaagtggcagggcgggattcacagctgcttacagcgagaaggggttggtttacatgagcatctgtggggagaatgaaaactgccctcctggcgtgggggcctgctttggacagaccaggattagcgtgggcaaggccaacaagaggctgagatacgtggaccaggtcctgcagctggtgtacaaggatgggtccccttgtccctccaaatccggcctgagctataagagtgtgatcagtttcgtgtgcaggcctgaggccgggccaaccaataggcccatgctcatctccctggacaagcagacatgcactctcttcttctcctggcacacgccgctggcctgcgagcaagcgaccgaatgttccgtgaggaatggaagctctattgttgacttgtctccccttattcatcgcactggtggttatgaggcttatgatgagagtgaggatgatgcctccgataccaaccctgatttctacatcaatatttgtcagccactaaatcccatgcacggagtgccctgtcctgccggagccgctgtgtgcaaagttcctattgatggtccccccatagatatcggccgggtagcaggaccaccaatactcaatccaatagcaaatgagatttacttgaattttgaaagcagtactccttgccaggaattcagttgtaaataaaattgaacctgctcaacagctgagggagactagaaatgatgggtccatatcctggtgcattgtcatacaattcaaacaatggtgcagctaccagcttgtaatttttagggactgcaaacaaggctttttcttgaagctgaaccagaaacaacttcttatgttccttaggctttgtaatatgtgcaggaatatatggatactgaggaggttcaaaatttggtctccaccagttaccaatgcaatcgtcaatgacccagtcttgcaaaactccatcctgacgacccagtatctctgtcattaagcgttttagtccttcaacttcatcttctcctgggttaagttcaccaccaggtagtttgaagaaagttgttcccagctgcagcagtaacacatggggtagccggtgctcatgtacaatcagaaccccttctacagtcctcctcattccaattttatcaaattcttccctcatgcgctgaaatctggctgcaacagagctgtccttctcgtagaggggctcttttgtaccaaaagtataattggtaagagggtacaggttgatggtgcgctccagggtgaggggcttcgtctgctggatgtacttgttgccgaactgagtgaccccccggggccagccggtctgcgagcgattgggcggtaccacagacatgctggcgagctccggcgctgacggcgagcagaaagtggcaggcagggtagactttccccgtgcgggaagcctcgtgccgaattc

SEQ ID NO: 8 - DNA (Genomic DNA), 4218 bp; double-stranded, linear
gaattcggcacgagaatggatcaacctcaacaacacgttaaagctagacgaaagaagtaatacacagtgtatgagtctcacatgaaatacccggatgtaaatccaaagaaacaggaagcagattggtggttgccagggacaagggcggtgggaggagaaaatggagagtaacgggactttacttttggagtgatgagaatgttttggagctagatagaagtggtggttgtacaccattgtggatgtactaccacttaattgttcacttaaaaagttaatttatgtgaattgcatcttaattaaaaacaaggataacattccaactcctggacattatccttcctttccatttgatgtcaggcccgtgttagaattctcatccggtttggtcactgcacttaagatgtggagaaattaggacgcacagttaagaggaaggataacactgattaaggtagtgcttttctaggtttcccctaaacaatttaacagatggatagtggcaccacttacgagatggaaaaaccagcggaaggaagatttgggggagaagttaagtttgtcttgggcctgtgttttgcaacctgagtgtaaaagacatatgttaagtcttcagtggcgaaacactaaaactagaaatggatcagaattttatctttggatgtgacttctcaaggatggtcttgtcacttcagtgcctggtcaaatgacaagatgggcaatcttttcctgaaggtccaagcacctgaacgtggcagggtgacccgattccgatttgcttagaacaatcctagttcatgcctattgtccctcatgtaattaatatcactctcaaaatgtctcattttgtgcaataaattctgcaacgtgatggcgcgactctcgcggcccgagcggccggaccttgtcttcgaggaagaggacctcccctatgaggaggaaatcatgcggaaccaattctctgtcaaatgctggcttcactacatcgagttcaaacagggcgccccgaagcccaggctcaatcagctatacgagcgggcactcaagctgctgccctgcagctacaaactctggtaccgatacctgaaggcgcgtcgggcacaggtgaagcatcgctgtgtgaccgaccctgcctatgaagatgtcaacaactgtcatgagagggcctttgtgttcatgcacaagatgcctcgtctgtggctagattactgccagttcctcatggaccaggggcgcgtcacacacacccgccgcaccttcgaccgtgccctccgggcactgcccatcacgcagcactctcgaatttggcccctgtatctgcgcttcctgcgctcacacccactgcctgagacagctgtgcgaggctatcggcgcttcctcaagctgagtcctgagagtgcagaggagtacattgagtacctcaagtcaagtgaccggctggatgaggccgcccagcgcctggccaccgtggtgaacgacgagcgtttcgtgtctaaggccggcaagtccaactaccagctgtggcacgagctgtgcgacctcatctcccagaatccggacaaggtacagtccctcaatgtggacgccatcatccgcgggggcctcacccgcttcaccgaccagctgggcaagctctggtgttctctcgccgactactacatccgcagcggccatttcgagaaggctcgggacgtgtacgaggaggccatccggacagtgatgaccgtgcgggacttcacacaggtgtttgacagctacgcccagttcgaggagagcatgatcgctgcaaagatggagaccgcctcggagctggggcgcgaggaggaggatgatgtggacctggagctgcgcctggcccgcttcgagcagctcatcagccggcggcccctgctcctcaacagcgtcttgctgcgccaaaacccacaccacgtgcacgagtggcacaagcgtgtcgccctgcaccagggccgcccccgggagatcatcaacacctacacagaggctgtgcagacggtggaccccttcaaggccacaggcaagccccacactctgtgggtggcgtttgccaagttttatgaggacaacggacagctggacgatgcccgtgtcatcctggagaaggccaccaaggtgaacttcaagcaggtggatgacctggcaagcgtgtggtgtcagtgcggagagctggagctccgacacgagaactacgatgaggccttgcggctgctgcgaaaggccacggcgctgcctgcccgccgggccgagtactttgatggttcagagcccgtgcagaaccgcgtgtacaagtcactgaaggtctggtccatgctcgccgacctggaggagagcctcggcaccttccagtccaccaaggccgtgtacgaccgcatcctggacctgcgtatcgcaacaccccagatcgtcatcaactatgccatgttcctggaggagcacaagtacttcgaggagagcttcaaggcgtacgagcgcggcatctcgctgttcaagtggcccaacgtgtccgacatctggagcacctacctgaccaaattcattgcccgctatgggggccgcaagctggagcgggcacgggacctgtttgaacaggctctggacggctgccccccaaaatatgccaagaccttgtacctgctgtacgcacagctggaggaggagtggggcctggcccggcatgccatggccgtgtacgagcgtgccaccagggccgtggagcccgcccagcagtatgacatgttcaacatctacatcaagcgggcggccgagatctatggggtcacccacacccgcggcatctaccagaaggccattgaggtgctgtcggacgagcacgcgcgtgagatgtgcctgcggtttgcagacatggagtgcaagctcggggagattgaccgcgcccgggccatctacagcttctgctcccagatctgtgacccccggacgaccggcgcgttctggcagacgtggaaggactttgaggtccggcatggcaatgaggacaccatcaaggaaatgctgcgtatccggcgcagcgtgcaggccacgtacaacacgcaggtcaacttcatggcctcgcagatgctcaaggtctcgggcagtgccacgggcaccgtgtctgacctggcccctgggcagagtggcatggacgacatgaagctgctggaacagcgggcagagcagctggcggctgaggcggagcgtgaccagcccttgcgcgcccagagcaagatcctgttcgtgaggagtgacgcctcccgggaggagctggcagagctggcacagcaggtcaaccccgaggagatccagctgggcgaggacgaggacgaggacgagatggacctggagcccaacgaggttcggctggagcagcagagcgtgccagccgcagtgtttgggagcctgaaggaagactgacccgtcccctcgtgccgaattcggcacgagcaagaccagcccccagatcatttgcctcaaaggttttccctcgaagtcacaaatgtttcaaggaatctcaaattttacaaagtttgaagtgtgggcattggtggcctgtggctgtgtcctctctctgtagctgttttctccctacatccctgaaaggaagttgagcctgctcctccatccgcagacctccctttccagcgcccagggcatggggtgctgtgagggcagcatgctaggtgtgaccgtgctcctggcctccaggcccgtgtccctctgtcctctagcccactaaggccctggcccatttgtgctaaacaggcagtcggacctagaaagagcagacaatctctctgggtcaccagtctggctaggagctggtctcctgactgggatccaggccttctcccctgcccatgtgaattcccaggggcagagcctgaaatgttgaacacagcactggccaaagagatgtcaccgtgggaaccgaggctctcttctcctcctgcctgctttcgtgggttcagagtagctgaggcttgtctgagaggagttggagtgctggttttcaccctggttggtgtgctttgctttgagggcacttagaaagcccagcccagcccttgctcctgccctgcacacagcggagcgacttttctaggtatgctcttgatttctgcagaagcagcaggtggcatggagccaagaggaagtgtgactgaaactgtccactcatagcccggctgccgtattgagagggct

SEQ ID NO: 9 - DNA (Genomic DNA), 1187 bp; double-stranded, linear
gagctcgcgcgcctgcaggtcgacactagtggatccaaagaattcggcacgagggaaactcaacggtgtacgagtggaggacagggacagagccctctgtggtggaacgaccccacctcgaggagcttcctgagcaggtggcagaagatgcgattgactggggcgactttggggtagaggcagtgtctgaggggactgactctggcatctctgccgaggctgctggaatcgactggggcatcttcccggaatcagattcaaaggatcctggaggtgatgggatagactggggagacgatgctgttgctttgcagatcacagtgctggaagcaggaacccaggctccagaaggtgttgccaggggcccagatgccctgacactgcttgaatacactgagacccggaatcagttccttgatgagctcatggagcttgagatcttcttagcccagagagcagtggagttgagtgaggaggcagatgtcctgtctgtgagccagttccagctggctccagccatcctgcagggccagaccaaagagaagatggttaccatggtgtcagtgctggaggatctgattggcaagcttaccagtcttcagctgcaacacctgtttatgatcctggcctcaccaaggtatgtggaccgagtgactgaattcctccagcaaaagctgaagcagtcccagctgctggctttgaagaaagagctgatggtgcagaagcagcaggaggcacttgaggagcaggcggctctggagcctaagctggacctgctactggagaagaccaaggagctgcagaagctgattgaagctgacatctccaagaggtacagcgggcgccctgtgaacctgatgggaacctctctgtgacaccctccgtgttcttgcctgcccatcttctccgcttttgggatgaagatgatagccagggctgttgttttggggcccttcaaggcaaaagaccaggctgactggaagatggaaagccacaggaaggaagcggcacctgatggtgatcttggcactctccatgttctctacaagaagctgtggtgattggccctgtggtctatcaggcgaaaaccacagattctccttctagttagtatagcgcaaaaagcttctcgagagtacttctagagcggccgcgggcccatcgattttccacccgggtggggtacc

SEQ ID NO: 10 - DNA (Genomic DNA), 3306 bp; double-stranded, linear
ccctcactaaagggaacaaaagctggagctcgcgcgcctgcaggtcgacactagtggatcgaaagttcgttacgccaagctcgaaattaactctgggctgacccataaacatttgtctgatctaggatatagttgcgtttcttgcgggcagcaatctggatgaggcggttgaggcactgggtggcctgctggatcaggacatcccagcggccagcatagttccgctgccggcgtaggcccatcacccgcatcttatccatgatggcattggtacccaggatgttgtacttcttggaagggttggaggctgcatgtttgatggcccatgtggtcttgccagcagcaggcaggcccaccatcatcagaatctcacattctgccttgctctttggtccaacggtgccccggatacgctcactaaggggaaggtgctggatgaaggtaaaccccgggaggacagaacagtagggctctgctctctgtccgaagttgaactccactgcgcaattcttcaccaggacatgaggatagagggcctgaccccccaaggcttccttctggattcggaaagcaatgcccatccactttccattcttggtaaaagacagttccacgtcatttccacattcaaaatccgcaaagcagccaatcaccggagagctctgcggtgctaggagagcggctgggcccgcagactggggggaaagctccgcagccgcagtgggccccaggatcaggccccgcgtggcctggagaagcccagtctgggctggagcgggagctggacagtgtggccttgcgttcgcccccgggagcgctgcgagtgtcgcggcctcgggtggatttgctgagcaccaatacctcacggttgccaacctggggttttagctcccttggttttaatcccctaggggcgggtgggggcacgggaggaaggatgggccagctgggtgcaatcctgctgtaagccagccattccttgatttcttagaattaactaaacggtcgcgccggaggccgcgggggccggagcggagcagccgcggctgaggttcccgagtcggccgctcggggctgcgctccgccgccgggaccccggcctctggccgcgccggctccggcctccgggggggccggggccgccgggacatggtgccagtcgcaccccttccccgccgccgctgagctcgccggccgcgcccgggctgggacgtccgagcgggaagatgttttccgccctgaagaagctggtggggtcggaccaggccccgggccgggacaagaacatccccgccgggctgcagtccatgaaccaggcgttgcagaggcgcttcgccaagggggtgcagtacaacatgaagatagtgatccggggagacaggaacacgggcaagacagcgctgtggcaccgcctgcagggccggccgttcgtggaggagtacatccccacacaggagatccaggtcaccagcatccactggagctacaagaccacggatgacatcgtgaaggttgaagtctgggatgtagtagacaaaggaaaatgcaaaaagcgaggcgacggcttaaagatggagaacgacccccaggagncggagtctgaaatggccctggatgctgagttcctggacgtgtacaagaactgcaacggggtggtcatgatgttcgacattaccaagcagtggaccttcaattacattctccgggagcttccaaaagtgcccacccacgtgccagtgtgcgtgctggggaactaccgggacatgggcgagcaccgagtcatcctgccggacgacgtgcgtgacttcatcgacaacctggacagacctccaggttcctcctacttccgctatgctgagtcttccatgaagaacagcttcggcctaaagtaccttcataagttcttcaatatcccatttttgcagcttcagagggagacgctgttgcggcagctggagacgaaccagctggacatggacgccacgctggaggagctgtcggtgcagcaggagacggaggaccagaactacggcatcttcctggagatgatggaggctcgcagccgtggccatgcgtccccactggcggccaacgggcagagcccatccccgggctcccagtcaccagtcctgcctgcacccgctgtgtccacggggagctccagccccggcacaccccagcccgccccacagctgcccctcaatgctgccccaccatcctctgtgccccctgtaccaccctcagaggccctgcccccacctgcgtgcccctcagcccccgccccacggcgcagcatcatctctaggctgtttgggacgtcacctgccaccgaggcagcccctccacctccagagccagtcccggccgcacagggcccagcaacggtccagagtgtggaggactttgttcctgacgaccgcctggaccgcagcttcctggaagacacaacccccgccagggacgagaagaaggtgggggccaaggctgcccagcaggacagtgacagtgatggggaggccctgggcggcaacccgatggtggcagggttccaggacgatgtggacctcgaagaccagccacgtgggagtcccccgctgcctgcaggccccgtccccagtcaagacatcactctttcgagtgaggaggaagcagaagtggcagctcccacaaaaggccctgccccagctccccagcagtgctcagagccagagaccaagtggtcctccataccagcttcgaagccacggagggggacagctcccacgaggaccgcagcacccccctggccaggcggtgtctctgttcgcacaggtccggagaagcgcagcagcaccaggccccctgctgagatggagccggggaagggtgagcaggcctcctcgtcggagagtgaccccgagggacccattgctgcacaaatgctgtccttcgtcatggatgaccccgactttgagagcgagggatcagacacacagcgcagggcggatgactttcccgtgcgagatgacccctccgatgtgactgacgaggatgagggccctgccgagccgcccccaccccccaagctccctctccccgccttcagactgaagaatgactcggacctcttcgggctggggctggaggaggccggacccaaggagagcagtgaggaaggtaaggagggcaaaaccccctctaaggagaagaaaaaaaaaacaaaaagcttctcgagagtacttctagagcggccgcgggcccatcgattttccacccgggtggggtaccaggtaagtgtacccaattcgccctatagtgagtcgtatt

SEQ ID NO: 11 - DNA, 20 bp; single-stranded, linear
tgcggggccagagtgggctg

SEQ ID NO: 12 - DNA, 20 bp; single-stranded, linear
gcagtcctggcctgcggatg

SEQ ID NO: 13 - DNA, 20 bp; single-stranded, linear
gtcgacaggagaattggttc

SEQ ID NO: 14 - DNA, 20 bp; single-stranded, linear
gcctgggttcggtgcgggac

SEQ ID NO: 15 - DNA, 20 bp; single-stranded, linear
tggtcgggtgtttgtgagtg

SEQ ID NO: 16 - DNA, 20 bp; single-stranded, linear
cctcttccgtctcctcagtg

SEQ ID NO: 17 - DNA, 20 bp; single-stranded, linear
ggattgctagtctcacagac

SEQ ID NO: 18 - DNA, 20 bp; single-stranded, linear
ttaagggtggctgaagggac

SEQ ID NO: 19 - DNA, 20 bp; single-stranded, linear
accttccctccctgtcacag

SEQ ID NO: 20 - DNA, 20 bp; single-stranded, linear
tggtcgggtgtttgtgagtg

SEQ ID NO: 21 - DNA, 20 bp; single-stranded, linear
acaccattccagaaattcag

SEQ ID NO: 22 - DNA, 20 bp; single-stranded, linear
aaactgcaggtggctgagtc

SEQ ID NO: 23 - DNA, 20 bp; single-stranded, linear
gtcctaatgttttcagggag

SEQ ID NO: 24 - DNA, 20 bp; single-stranded, linear
aaaacctatggttacaattc

SEQ ID NO: 25 - DNA, 20 bp; single-stranded, linear
tcctagacatggttcaagtg

SEQ ID NO: 26 - DNA, 20 bp; single-stranded, linear
gatataattagttctccatc

SEQ ID NO: 27 - DNA, 20 bp; single-stranded, linear
atgcctgttccaggctgcac

SEQ ID NO: 28 - DNA, 20 bp; single-stranded, linear
ggacggcgacctccacccac

SEQ ID NO: 29 - DNA, 20 bp; single-stranded, linear
gggctcctccgacgcctgag

SEQ ID NO: 30 - DNA, 20 bp; single-stranded, linear
agtctagccctggccttgac

SEQ ID NO: 31 - DNA, 20 bp; single-stranded, linear
gtcactggggactccggcag

SEQ ID NO: 32 - DNA, 20 bp; single-stranded, linear
cagctttccctgggcacatg

SEQ ID NO: 33 - DNA, 20 bp; single-stranded, linear
cacagctgtctcaagcccag

SEQ ID NO: 34 - DNA, 20 bp; single-stranded, linear
actgttccccctacatgatg

SEQ ID NO: 35 - DNA, 20 bp; single-stranded, linear
atcatatcctcttgctggtc

SEQ ID NO: 36 - DNA, 20 bp; single-stranded, linear
gttcccagagcttgtctgtg

SEQ ID NO: 37 - DNA, 20 bp; single-stranded, linear
gtttggcagactcatagttg

SEQ ID NO: 38 - DNA, 20 bp; single-stranded, linear
tagcagggagccatgacctg

SEQ ID NO: 39 - DNA, 20 bp; single-stranded, linear
cttggcgccagaagcgagag

SEQ ID NO: 40 - DNA, 20 bp; single-stranded, linear
cctctctctctctctctctc

SEQ ID NO: 41 - DNA, 20 bp; single-stranded, linear
tccccgctgattccgccaag

SEQ ID NO: 42 - DNA, 20 bp; single-stranded, linear
ctttttgaattcggcacgag

SEQ ID NO: 43 - DNA, 20 bp; single-stranded, linear
cccctggtccgcaccagttc

SEQ ID NO: 44 - DNA, 20 bp; single-stranded, linear
gagaagggtcggggcggcag

SEQ ID NO: 45 - DNA, 20 bp; single-stranded, linear
aaatcacatcgcgtcaacac

SEQ ID NO: 46 - DNA, 20 bp; single-stranded, linear
taagagagtcatagttactc

SEQ ID NO: 47 - DNA, 20 bp; single-stranded, linear
gctctagaagtactctcgag

SEQ ID NO: 48 - DNA, 20 bp; single-stranded, linear
actctggccatcaggagatc

SEQ ID NO: 49 - DNA, 20 bp; single-stranded, linear
caggcgttgtagatgttctg

SEQ ID NO: 50 - DNA, 20 bp; single-stranded, linear
agtggcaggcagaagtaatg

SEQ ID NO: 51 - DNA, 20 bp; single-stranded, linear
ggttggagaactggatgtag

SEQ ID NO: 52 - DNA, 20 bp; single-stranded, linear
ctattcagatgcaacgccag

SEQ ID NO: 53 - DNA, 20 bp; single-stranded, linear
ccatggcacacagagcagac

SEQ ID NO: 54 - DNA, 20 bp; single-stranded, linear
gctaccatgcagagacacag

SEQ ID NO: 55 - DNA, 20 bp; single-stranded, linear
caggctgacaagaaaatcag

SEQ ID NO: 56 - DNA, 20 bp; single-stranded, linear
ggcacgcatagaggagagac

SEQ ID NO: 57 - DNA, 20 bp; single-stranded, linear
tgggtgatgcctttgctgac

SEQ ID NO: 58 - DNA, 20 bp; single-stranded, linear
aaaacaagatcaaggtgatg

SEQ ID NO: 59 - DNA, 20 bp; single-stranded, linear
ttgcccacattgctatggtg

SEQ ID NO: 60 - DNA, 20 bp; single-stranded, linear
gaccaagatcagaagtagag

SEQ ID NO: 61 - DNA, 20 bp; single-stranded, linear
cccctgggccaatgatgttg

SEQ ID NO: 62 - DNA, 19 bp; single-stranded, linear
tcttcccaccatagcaatg

SEQ ID NO: 63 - DNA, 20 bp; single-stranded, linear
tggtcttggtgaccaatgtg

SEQ ID NO: 64 - DNA, 20 bp; single-stranded, linear
acacctcggtgacccctgtg

SEQ ID NO: 65 - DNA, 20 bp; single-stranded, linear
tctccaagttcggcacagtg

SEQ ID NO: 66 - DNA, 20 bp; single-stranded, linear
acatgggctgcactcacgac

SEQ ID NO: 67 - DNA, 20 bp; single-stranded, linear
gatcctctgaacctgcagag

SEQ ID NO: 68 - DNA, 20 bp; single-stranded, linear
ggaaatgaggtggggcgatc

SEQ ID NO: 69 - DNA, 20 bp; single-stranded, linear
ctttgccttggacaaggatg

SEQ ID NO: 70 - DNA, 20 bp; single-stranded, linear
gcacctgccattgggggtag

SEQ ID NO: 71 - DNA, 20 bp; single-stranded, linear
ggtggaagccattgacggtg

SEQ ID NO: 72 - DNA, 20 bp; single-stranded, linear
tgcgtctctcgtcgctgctg

SEQ ID NO: 73 - DNA, 20 bp; single-stranded, linear
gcggaaactctgtggtgctg

SEQ ID NO: 74 - DNA, 20 bp; single-stranded, linear
aggattgccttcctctactg

SEQ ID NO: 75 - DNA, 20 bp; single-stranded, linear
tgtctgtttcaccagggcag

SEQ ID NO: 76 - DNA, 20 bp; single-stranded, linear
ccagtgcctctatgcatgtc

SEQ ID NO: 77 - DNA, 20 bp; single-stranded, linear
aggaagcccacgcacaccac

SEQ ID NO: 78 - DNA, 20 bp; single-stranded, linear
ccctttgttccctgatcttc

SEQ ID NO: 79 - DNA, 20 bp; single-stranded, linear
cgctcgggatccaggtcatc

SEQ ID NO: 80 - DNA, 20 bp; single-stranded, linear
tcgaggttcagagcgtagtg

SEQ ID NO: 81 - DNA, 20 bp; single-stranded, linear
tcttggatctctggcacctc

SEQ ID NO: 82 - DNA, 20 bp; single-stranded, linear
ccatcagagtgaaggaggag

SEQ ID NO: 83 - DNA, 20 bp; single-stranded, linear
ccatcttccactggtcagag

SEQ ID NO: 84 - DNA, 20 bp; single-stranded, linear
ctccttctcttggatctctg

SEQ ID NO: 85 - DNA, 20 bp; single-stranded, linear
ttacttcagcactgttagtc

SEQ ID NO: 86 - DNA, 20 bp; single-stranded, linear
agggaggtagctcaaagctc

SEQ ID NO: 87 - DNA, 20 bp; single-stranded, linear
tgggtccacagttcgcacag

SEQ ID NO: 88 - DNA, 20 bp; single-stranded, linear
caactctgtgatggctccag

SEQ ID NO: 89 - DNA, 20 bp; single-stranded, linear
agcagggttctgttcaagac

SEQ ID NO: 90 - DNA, 20 bp; single-stranded, linear
ccattgggtgctagtctctc

SEQ ID NO: 91 - DNA, 20 bp; single-stranded, linear
cagccatgctgtcccagcag

SEQ ID NO: 92 - DNA, 20 bp; single-stranded, linear
ctggacctgaggtagcgctg

SEQ ID NO: 93 - DNA, 20 bp; single-stranded, linear
ataaccaccctgaggcactg

SEQ ID NO: 94 - DNA, 20 bp; single-stranded, linear
cctgcaggtcgacactagtg

SEQ ID NO: 95 - DNA, 20 bp; single-stranded, linear
aattggaatgaggaggactg

SEQ ID NO: 96 - DNA, 20 bp; single-stranded, linear
gctctagaagtactctcgag

SEQ ID NO: 97 - DNA, 20 bp; single-stranded, linear
attgtatgacaatgcaccag

SEQ ID NO: 98 - DNA, 20 bp; single-stranded, linear
tccacagagggcttcatcac

SEQ ID NO: 99 - DNA, 20 bp; single-stranded, linear
cctgactggcctaagcacag

SEQ ID NO: 100 - DNA, 20 bp; single-stranded, linear
aagcctcataaccaccagtg

SEQ ID NO: 101 - DNA, 20 bp; single-stranded, linear
tgtcaacggtgacaagtgtg

SEQ ID NO: 102 - DNA, 20 bp; single-stranded, linear
ttgtacaccagctgcaggtc

SEQ ID NO: 103 - DNA, 20 bp; single-stranded, linear
gggtgtggtgcagatgagtc

SEQ ID NO: 104 - DNA, 20 bp; single-stranded, linear
atcacactcttatagctcag

SEQ ID NO: 105 - DNA, 20 bp; single-stranded, linear
gtgggaagctttcctcagac

SEQ ID NO: 106 - DNA, 20 bp; single-stranded, linear
tgatgaacatgggcctggag

SEQ ID NO: 107 - DNA, 20 bp; single-stranded, linear
cattgtggatgtactaccac

SEQ ID NO: 108 - DNA, 20 bp; single-stranded, linear
tgtgttttgcaacctgagtg

SEQ ID NO: 109 - DNA, 20 bp; single-stranded, linear
atagtggcaccacttacgag

SEQ ID NO: 110 - DNA, 20 bp; single-stranded, linear
aattctgcaacgtgatggcg

SEQ ID NO: 111 - DNA, 20 bp; single-stranded, linear
cacaagatgcctcgtctgtg

SEQ ID NO: 112 - DNA, 20 bp; single-stranded, linear
aatccggacaaggtacagtc

SEQ ID NO: 113 - DNA, 20 bp; single-stranded, linear
gcacgagtggcacaagcgtg

SEQ ID NO: 114 - DNA, 20 bp; single-stranded, linear
gcaagcgtgtggtgtcagtg

SEQ ID NO: 115 - DNA, 20 bp; single-stranded, linear
tgtttgaacaggctctggac

SEQ ID NO: 116 - DNA, 20 bp; single-stranded, linear
cggcatggcaatgaggacac

SEQ ID NO: 117 - DNA, 20 bp; single-stranded, linear
aggacgagatggacctccag

SEQ ID NO: 118 - DNA, 20 bp; single-stranded, linear
ccctctgtcctctagcccac

SEQ ID NO: 119 - DNA, 20 bp; single-stranded, linear
tcttgaggggactgactctg

SEQ ID NO: 120 - DNA, 20 bp; single-stranded, linear
tgagtgaggaggcagatgtc

SEQ ID NO: 121 - DNA, 20 bp; single-stranded, linear
tggctttgaagaaagagctg

SEQ ID NO: 122 - DNA, 20 bp; single-stranded, linear
gcaaaagaccaggctgactg

SEQ ID NO: 123 - DNA, 20 bp; single-stranded, linear
tgcagctccttggtcttctc

SEQ ID NO: 124 - DNA, 20 bp; single-stranded, linear
gattcacagtcccaaggctc

SEQ ID NO: 125 - DNA, 20 bp; single-stranded, linear
atctggatgaggcggttgag

SEQ ID NO: 126 - DNA, 20 bp; single-stranded, linear
ggtcactctccgacgaggag

SEQ ID NO: 127 - DNA, 20 bp; single-stranded, linear
ggatccaaagttcgtctctg

SEQ ID NO: 128 - DNA, 20 bp; single-stranded, linear
cgctgtgtgtctgatccctc

SEQ ID NO: 129 - DNA, 20 bp; single-stranded, linear
atgaaggtaaaccccgggag

SEQ ID NO: 130 - DNA, 20 bp; single-stranded, linear
tggtctctggctctgagcac

SEQ ID NO: 131 - DNA, 20 bp; single-stranded, linear
gcctggagaagcccagtctg

SEQ ID NO: 132 - DNA, 20 bp; single-stranded, linear
cacactctggaccgttgctg

SEQ ID NO: 133 - DNA, 20 bp; single-stranded, linear
aaagctccgcagccgcagtg

SEQ ID NO: 134 - DNA, 20 bp; single-stranded, linear
tcttccaggaagctgcggtc

SEQ ID NO: 135 - DNA, 20 bp; single-stranded, linear
gatggtggggcagcattgag

SEQ ID NO: 136 - DNA, 20 bp; single-stranded, linear
gtcaccagtggtgcctgcag

SEQ ID NO: 137 - DNA, 20 bp; single-stranded, linear
acctcacggttgccaacctg

SEQ ID NO: 138 - DNA, 20 bp; single-stranded, linear
cgcaacagcgtctccctctg

SEQ ID NO: 139 - DNA, 20 bp; single-stranded, linear
agtaccttcataagttcttc

SEQ ID NO: 140 - DNA, 20 bp; single-stranded, linear
tcccagacttcaaccttcac

SEQ ID NO: 141 - DNA, 20 bp; single-stranded, linear
aaacatcttcccggtcggac

SEQ ID NO: 142 - DNA, 20 bp; single-stranded, linear
gctgagcacctttacctcac

SEQ ID NO: 143 - DNA, 20 bp; single-stranded, linear
gacgtccgtccgggaagatg

SEQ ID NO: 144 - DNA, 20 bp; single-stranded, linear
acacaggagatgcaggtcac

SEQ ID NO: 145 - DNA, 20 bp; single-stranded, linear
gagtcttccatgaagaacag

SEQ ID NO: 146 - DNA, 20 bp; single-stranded, linear
gcagtgaggaaggtaaggag

SEQ ID NO: 147 - DNA (Genomic DNA), 4047 bp; double-stranded, linear
  Coding Sequence 378...1799
ggatccaaaggacgcccccgccgacaggagaattggttcccgggcccgcggcgatgcccccccggtagctcgggcccgtggtcgggtgtttgtgagtgtttctatgtgggagaaggaggaggaggaggaagaagaagcaacgatttgtcttctcggctggtctccccccggctctacatgttccccgcactgaggagacggaagaggagccgtagccgccccccctcccggcccggattatagtctctcgccacagcggcctcggcctccccttggattcagacgccgattcgcccagtgtttgggaaatgggaagtaatgacagctggcacctgaactaagtacttttataggcaacaccattccagaaattcaggatgaatggggatatgccccatgtccccattactactcttgcggggattgctagtctcacagacctcctgaaccagctgcctcttccatctcctttacctgctacaactacaaagagccttctctttaatgcacgaatagcagaagaggtgaactgccttttggcttgtagggatgacaatttggtttcacagcttgtccatagcctcaaccaggtatcaacagatcacatagagttgaaagataaccttggcagtgatgacccagaaggtgacataccagtcttgttgcaggccgtcctggcaaggagtcctaatgttttcagggagaaaagcatgcagaacagatatgtacaaagtggaatgatgatgtctcagtataaactttctcagaattccatgcacagtagtcctgcatcttccaattatcaacaaaccactatctcacatagcccctccagccggtttgtgccaccacagacaagctctgggaacagatttatgccacagcaaaatagcccagtgcctagtccatacgccccacaaagccctgcaggatacatgccatattcccatccttcaagttacacaacacatccacagatgcaacaagcatcggtatcaagtcccattgttgcaggtggtttgagaaacatacatgataataaagtttctggtccgttgtctggcaattcagctaatcatcatgctgataatcctagacatggttcaagtgaggactacctacacatggtgcacaggctaagtagtgacgatggagattcttcaacaatgaggaatgctgcatcttttcccttgagatctccacagccagtatgctcccctgctggaagtgaaggaactcctaaaggctcaagaccacctttaatcctacaatctcagtctctaccttgttcatcacctcgagatgttccaccagatatcttgctagattctccagaaagaaaacaaaagaagcagaagaaaatgaaattaggcaaggatgaaaaagagcagagtgagaaagcggcaatgtatgatataattagttctccatccaaggactctactaaacttacattaagactttctcgtgtaaggtcttcagacatggaccagcaagaggatatgatttctggtgtggaaaatagcaatgtttcagaaaatgatattccttttaatgtgcagtacccaggacagacttcaaaaacacccattactccacaagatataaaccgcccactaaatgctgctcaatgtttgtcgcagcaagaacaaacagcattccttccagcaaatcaagtgcctgttttacaacagaacacttcagttgctgcaaaacaaccccagaccaatagtcacaaaaccttggtgcagcctggaacaggcatagaggtctcagcagagctgcccaaggacaagacctaagatccagcagggaactatgtagtcaccccgagaggcccagctctctccgtgagctctgggcctagggtgggggtggttgttggttctgcgcgcactgttccccctacatgatgggtccatcccagttggcttctctcactcgcttcctcctgtggagaagcctgtccaggtgtcactgcctccaggaagctgtctctgatttctccagttgaacagtgagatttgccacacctcacatgcatcgctcttgtccctggaattgtaaccataggttttcctgtctcctggaggacaaggatgagggctttccacttgagtctccctggtggagcccagctcctgacatacctggtaaaagttctcaagagaagaacatggaggaggaatgtggataacaaccctggctgcctgtgtgttccaagctaggaagatgtaatgtccccacaaacggggtaaatggcttgcctgcgtcacagctgtctcaagcccaggccctgggcgccagcccaagcccaaggactaggtccagagccacacagcgccaggccacatccgcctcacctgggaccctttgtggggtacagtctccggccccacccagacctcctgaaggagagaccccatggcaaggactcagccacctgcagtttcataagcccccagtgggttcctaggcatgaagaccaccggttagaggctgaactggcaggaacctgtctccagccccttctcaccccagccgggccctgcctcagaggcagcacccaggacgtggccatgacccgtggactccactcaatccctcttctccaggagccatgcaaagtgtcagccagccaggcccctggaaggcagtcatcacctcttaaggcattgtgggtgtcggtcctgcaactgccaggtgcagcacacgacccgtgtccggtgttcgatagcagggagccatgacctggcaacgattccacgctcaaaggggcacccggggggccctgggtcggggcggatcagctttccctgggcacatctgcctcattccagatctccagggctcatgtctgtgacagggagggaaggctctgccctggccttccgtcagctctgccagtgcaggctgggcagcctgggctttagagctggcttctgcccacactttctccgtgaaaggaaaacaactatgagtctgccaaacgcatctcagatgcgttttaaaaaattctggtccccgctctctgtcccatcatccgcctcggggacttcctctctccgtggttctcaccccatactctgtcactgccacattttcacctgggcctggcctttgtctccacctgaaactcctgaaaatcttgaaatggatttctaggtcactggggactccggcagcacattcggcttcagaataaagggcgcccgcggtcccccagcacctccccaagccacacccctagcttccctccctatccctgcagcctgagggtcccttcagccacccttaagtccccacctgggctcctgccccgcccctggctagcagcgccttctccaccggggccccctctgctcacagagccccctcacctccctggggatgaggggccaggccatgaccctgaaagtctagccctggccttgacctcccaggagcgccctccccgccctctcccggccccggccccgtcctctgctgctggcctctgggtcgtgccccgcagactgagctgcgcttgggggtcctggcggcctgggccgtcccgcaccgaacccaggcggtcggagcccggcgggaaggcgcgaggtccttctgggggctcctccgacgcctgagggcgctgcttccccgcggccgccccgggtttctgcggagccggggcctccgctctcgggtgacccggtgagacccccggggaggccgctggggaggcgcgggctctgctcccgggtcccaaacgcactggctgcccctcaggagggacggcgacctccacccacggcgctggcgcccgcacggccgctcctcccgctcccgcagcctggacgcctcccgaggccgccccgccgggccccacgcgcggccccatccgcaggccaggactgccttcccggagctggcggcccccagcctggaggagccggccccagacgccctcccagccctccccagcccactctggccccgcagcccccgcctggtccgagtgcgggtctctggccccggcctttcccggggaaggaaagcaaaaagctt

SEQ ID NO: 148 - protein, 474 aa
  fragment type: internal
MNGDMPHVPITTLAGIASLTDLLNQLPLPSPLPATTTKSLLFNARIAEEVNCLLACRDDNLVSQLVHSLNQVSTDHIELKDNLGSDDPEGDIPVLLQAVLARSPNVFREKSMQNRYVQSGMMMSQYKLSQNSMHSSPASSNYQQTTISHSPSSRFVPPQTSSGNRFMPQQNSPVPSPYAPQSPAGYMPYSHPSSYTTHPQMQQASVSSPIVAGGLRNIHDNKVSGPLSGNSANHHADNPRHGSSEDYLHMVHRLSSDDGDSSTMRNAASFPLRSPQPVCSPAGSEGTPKGSRPPLILQSQSLPCSSPRDVPPDILLDSPERKQKKQKKMKLGKDEKEQSEKAAMYDIISSPSKDSTKLTLRLSRVRSSDMDQQEDMISGVENSNVSENDIPFNVQYPGQTSKTPITPQDINRPLNAAQCLSQQEQTAFLPANQVPVLQQNTSVAAKQPQTNSHKTLVQPGTGIEVSAELPKDKT

SEQ ID NO: 149 - DNA (Genomic DNA), 2998 bp; double-stranded, linear
  Coding Sequence 26...799
aagctttttgaattcggcacgagatgctacacaggctatatttgaaatactggagaaatcctggttgccccagaattgtacactggttgatatgaagattgaatttggtgttgatgtaaccaccaaagaaattgttcttgctgatgttattgacaatgattcctggagactctggccatcaggagatcgaagccaacagaaagacaaacagtcttatcgggacctcaaagaagtaactcctgaagggctccaaatggtaaagaaaaactttgagtgggttgcagagagagtagagttgcttttgaaatcagaaagtcagtgcagggttgtagtgttgatgggctctacttctgatcttggtcactgtgaaaaaatcaagaaggcctgtggaaattttggcattccatgtgaacttcgagtaacatctgcgcataaaggaccagatgaaactctgaggattaaagctgagtatgaaggggatggcattcctactgtatttgtggcagtggcaggcagaagtaatggtttgggaccagtgatgtctgggaacactgcatatccagttatcagctgtcctcccctcacaccagactggggagttcaggatgtgtggtcttctcttcgactacccagtggtcttggctgttcaaccgtactttctccagaaggatcagctcaatttgctgctcagatatttgggttaagcaaccatttggtatggagcaaactgcgagcaagcattttgaacacatggatttccttgaagcaggctgacaagaaaatcagagaatgtaatttataagaaagaatgccattgaattttttaggggaaaaactacaaatttctaatttagctgaaggaaaatcaagcaagatgaaaaggtaattttaaattagagaacacaaataaaatgtattagtgaataaatggtgagggtaggcctattcagatgcaaggccagcaatggggctccccattatccccacccctttggtcccagtccccttctctgcaatgggcacgcatagaggagagacaaagggtattagacgcaacatcattggcccaggggagtccgagaagagctgccattggctgacagggcattttcaggctctgtcattggtcagggagcacaccccagcctgaagagtgatgccattggccagggagtggttttgtcatagccgttggctgtgaagtggaaggaaaagatctgggaatgaagccctgtggccaggaagatagacagggcagcaacttctgggcctccaggccctcttcccaccatagcaatgtgggcaaaactggtgtcaggccccagccagaaaaaggagcccaagccagagggcaagtgacaaaggatgtaccatgtccaatctcccacaccctggggctgcccttcccaatgtctttcttgatagccaagttgggctgggagcagctcactgctcctctagccaggagggtttctcagctcctggaggccgcagcttgatgttgaactgctgcagggtctgctccagctgtttctggttcccagcaaagtaggcggacacagcattgtggaagagcagcagctgcttgtgcatcaccttgatcttgttttcttccaggaacttgagcttgatggccacatctccccgcagcttctcatacttgtcccgatgggcctggaaagtggcctgggcactctcaagtcgaccacgtgtccctgcatcccgggggcctagactcagctcctctaagtctgttcggtaggcatcatattccagcctggcagcctcatactgtttcacagtcatgagcgtgtcttccatggtcttggtgaccaatgtgttgatgctagagacaaagaagttcacggctcctagcagcgtttccccattcttgcatagtagtttctgtgtctctgcattgtagccaaattcctcctgaagctctggggacttctggctgaggtcagcaaaggcatcacccagtgcatgctgggtctgcagcaggctgtagaggtgggctgtcagtgcccggcccagctgcaggacactctcatacttgcgcttcgtctcacgcagcaactcaatctgcagctctagctccaggattccggcgcctccactccgtcccccgcgggtctgctctgtgtgccatggacggcattgtcccagatatagccgttggtacaaagcggggatctgacgagcttttctctacttgtgtcactaacggaccgtttatcatgagcagcaactcggcttctgcagcaaacggaaatgacagcaagaagttcaaaggtgacagccgaagtgcaggcgtcccctctagagtgatccacatccggaagctccccatcgacgtcacggagggggaagtcatctccctggggctgccctttgggaaggtcaccaacctcctgatgctgaaggggaaaaaccaggccttcatcgagatgaacacggaggaggctgccaataccatggtgaactactacacctcggtgacccctgtgctgcgcggccagcccatctacatccagttctccaaccacaaggagctgaagaccgacagctctcccaaccaggcgcgggcccaggcggccctgcaggcggtgaactcggtccagtcggggaacctggccttggctgcctcggcggcggccgtggatgcagggatggcgatggccgggcagagccccgtgctcaggatcatcgtggagaacctcttctaccctgtgaccctggatgtgctgcaccagattttctccaagttcggcacagtgttgaagatcatcaccttcaccaagaacaaccagttccaggccctgctgcagtatgcggaccccgtgagcgcccagcacgccaagctgtcgctggacgggcagaacatctacaacgcctgctgcacgctgcgcatcgacttttccaagctcaccagcctcaacgtcaagtacaacaatgacaagagccgtgactacctcgtgccgaattctttggatcc

SEQ ID NO: 150 - protein, 258 aa
  fragment type: internal
ATQAIFEILEKSWLPQNCTLVDMKIEFGVDVTTKEIVLADVIDNDSWRLWPSGDRSQQKDKQSYRDLKEVTPEGLQMVKKNFEWVAERVELLLKSESQCRVVVLMGSTSDLGHCEKIKKACGNFGIPCELRVTSAHKGPDETLRIKAEYEGDGIPTVFVAVAGRSNGLGPVMSGNTAYPVISCPPLTPDWGVQDVWSSLRLPSGLGCSTVLSPEGSAQFAAQIFGLSNHLVWSKLRASILNTWISLKQADKKIRECNL

SEQ ID NO: 151 - protein, 1038 aa
IQRFGTSGHIMNLQAQPKAQNKRKRCLFGGQEPAPKEQPPPLQPPQQSIRVKEEQYLGHEGPGGAVSTSQPVELPPPSSLALLNSVVYGPERTSAAMLSQQVASVKWPNSVMAPGRGPERGGGGGVSDSSWQQQPGQPPPHSTWNCHSLSLYSATKGSPHPGVGVPTYYNHPEALKREKAGGPQLDRYVRPMMPQKVQLEVGRPQAPLNSFHAAKKPPNQSLPLQPFQLAFGHQVNRQVFRQGPPPPNPVAAFPPQKQQQQQQPQQQQQQQQAALPQMPLFENFYSMPQQPSQQPQDFGLQPAGPLGQSHLAHHSMAPYPFPPNPDMNPELRKALLQDSAPQPALPQVQIPFPRRSRRLSKEGILPPSALDGAGTQPGQEATGNLFLHHWPLQQPPPGSLGQPHPEALGFPLELRESQLLPDGERLAPNGREREAPAMGSEEGMRAVSTGDCGQVLRGGVIQSTRRRRRASQEANLLTLAQKAVELASLQNAKDGSGSEEKRKSVLASTTKCGVEFSEPSLATKRAREDSGMVPLIIPVSVPVRTVDPTEAAQAGGLDEDGKGLEQNPAEHKPSVIVTRRRSTRIPGTDAQAQAEDMNVKLEGEPSVRKPKQRPRPEPLIIPTKAGTFIAPPVYSNITPYQSHLRSPVRLADHPSERSFELPPYTPPPILSPVREGSGLYFNAIISTSTIPAPPPITPKSAHRTLLRTNSAEVTPPVLSVMGEATPVSIEPRINVGSRFQAEIPLMRDRALAAADPHKADLVWQPWEDLESSREKQRQVEDLLTAACSSIFPGAGTNQELALHCLHESRGDILETLNKLLLKKPLRPHNHPLATYHYTGSDQWKMAERKLFNKGIAIYKKDFFLVQKLIQTKTVAQCVEFYYTYKKQVKIGRNGTLTFGDVDTSDEKSAQEEVEVDIKTSQKFPRVPLPRRESPSEERLEPKREVKEPRKEGEEEVPEIQEKEEQEEGRERSRRAAAVKATQTLQANESASDILILRSHESNAPGSAGGQASEKPREGTGKSRRALPFSEKKKKKQKA

SEQ ID NO: 152 - protein, 849 aa
IRHEVSFLWNTEAACPIQTTTDTDQACSIRDPNSGFVFNLNPLNSSQGYNVSGIGKIFMFNVCGTMPVCGTILGKPASGCEAETQTEELKNWKPARPVGIEKSLQLSTEGFITLTYKGPLSAKGTADAFIVRFVCNDDVYSGPLKFLHQDIDSGQGIRNTYFEFETALACVPSPVDCQVTDLAGNEYDLTGLSTVRKPWTAVDTSVDGRKRTFYLSVCNPLPYIPGCQGSAVGSCLVSEGNSWNLGVVQMSPQAAANGSLSIMYVNGDKCGNQRFSTRITFECAQISGSPAFQLQDGCEYVFIWRTVEACPVVRVEGDNCEVKDPRHGNLYDLKPLGLNDTIVSAGEYTYYFRVCGKLSSDVCPTSDKSKVVSSCQEKREPQGFHKVAGLLTQKLTYENGLLKMNFTGGDTCHKVYQRSTAIFFYCDRGTQRPVFLKETSDCSYLFEWRTQYACPPFDLTECSFKDGAGNSFDLSSLSRYSDNWEAITGTGDPEHYLINVCKSLAPQAGTEPCPPEAAACLLGGSKPVNLGRVRDGPQWRDGIIVLKYVDGDLCPDGIRKKSTTIRFTCSESQVNSRPMFISAVEDCEYTFAWPTATACPMKSNEHDDCQVTNPSTGHLFDLSSLSGRAGFTAAYSEKGLVYMSICGENENCPPGVGACFGQTRISVGKANKRLRYVDQVLQLVYKDGSPCPSKSGLSYKSVISFVCRPEAGPTNRPMLISLDKQTCTLFFSWHTPLACEQATECSVRNGSSIVDLSPLIHRTGGYEAYDESEDDASDTNPDFYINICQPLNPMHGVPCPAGAAVCKVPIDGPPIDIGRVAGPPILNPIANEIYLNFESSTPCQEFSCK

SEQ ID NO: 153 - protein, 852 aa
MARLSRPERPDLVFEEEDLPYEEEIMRNQFSVKCWLHYIEFKQGAPKPRLNQLYERALKLLPCSYKLWYRYLKARRAQVKHRCVTDPAYEDVNNCHERAFVFMHKMPRLWLDYCQFLMDQGRVTHTRRTFDRALRALPITQHSRIWPLYLRFLRSHPLPETAVRGYRRFLKLSPESAEEYIEYLKSSDRLDEAAQRLATVVNDERFVSKAGKSNYQLWHELCDLISQNPDKVQSLNVDAIIRGGLTRFTDQLGKLWCSLADYYIRSGHFEKARDVYEEAIRTVMTVRDFTQVFDSYAQFEESMIAAKMETASELGREEEDDVDLELRLARFEQLISRRPLLLNSVLLRQNPHHVHEWHKRVALHQGRPREIINTYTEAVQTVDPFKATGKPHTLWVAFAKFYEDNGQLDDARVILEKATKVNFKQVDDLASVWCQCGELELRHENYDEALRLLRKATALPARRAEYFDGSEPVQNRVYKSLKVWSMLADLEESLGTFQSTKAVYDRILDLRIATPQIVINYAMFLEEHKYFEESFKAYERGISLFKWPNVSDIWSTYLTKFIARYGGRKLERARDLFEQALDGCPPKYAKTLYLLYAQLEEEWGLARHAMAVYERATRAVEPAQQYDMFNIYIKRAAEIYGVTHTRGIYQKAIEVLSDEHAREMCLRFADMECKLGEIDRARAIYSFCSQICDPRTTGAFWQTWKDFEVRHGNEDTIKEMLRIRRSVQATYNTQVNFMASQMLKVSGSATGTVSDLAPGQSGMDDMKLLEQRAEQLAAEAERDQPLRAQSKILFVRSDASREELAELAQQVNPEEIQLGEDEDEDEMDLEPNEVRLEQQSVPAAVFGSLKED

SEQ ID NO: 154 - protein, 693 aa
MFSALKKLVGSDQAPGRDKNIPAGLQSMNQALQRRFAKGVQYNMKIVIRGDRNTGKTALWHRLQGRPFVEEYIPTQEIQVTSIHWSYKTTDDIVKVEVWDVVDKGKCKKRGDGLKMENDPQEXESEMALDAEFLDVYKNCNGVVMMFDITKQWTFNYILRELPKVPTHVPVCVLGNYRDMGEHRVILPDDVRDFIDNLDRPPGSSYFRYAESSMKNSFGLKYLHKFFNIPFLQLQRETLLRQLETNQLDMDATLEELSVQQETEDQNYGIFLEMMEARSRGHASPLAANGQSPSPGSQSPVLPAPAVSTGSSSPGTPQPAPQLPLNAAPPSSVPPVPPSEALPPPACPSAPAPRRSIISRLFGTSPATEAAPPPPEPVPAAQGPATVQSVEDFVPDDRLDRSFLEDTTPARDEKKVGAKAAQQDSDSDGEALGGNPMVAGFQDDVDLEDQPRGSPPLPAGPVPSQDITLSSEEEAEVAAPTKGPAPAPQQCSEPETKWSSIPASKPRRGTAPTRTAAPPWPGGVSVRTGPEKRSSTRPPAEMEPGKGEQASSSESDPEGPIAAQMLSFVMDDPDFESEGSDTQRRADDFPVRDDPSDVTDEDEGPAEPPPPPKLPLPAFRLKNDSDLFGLGLEEAGPKESSEEGKEGKTPSKEKKKKTKSFSRVLLERPRAHRFSTRVGYQVSVPNSPYSESY

(19) United States
(12) Patent Application Publication
(10) Pub. No.: US 2005/0191661 A1
(43) Pub. Date: Sep. 1, 2005

(54) Treatment of inflammatory disease by cleaving TNF receptors

(76) Inventors: Tetsuya Gatanaga, Irvine, CA (US); Gale A. Granger, Laguna Beach, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/012,047

(22) Filed: Dec. 13, 2004

Related U.S. Application Data
(63) Continuation-in-part of application No. 09/562,912, filed on May 2, 2000, now Pat. No. 6,858,402.
Application No. 09/562,912 is a division of application No. 08/964,747, filed on Nov. 5, 1997, now Pat. No. 6,569,664.
(63) Continuation of application No. 10/967,092, filed on Oct. 15, 2004.
Application No. 10/967,092 is a continuation of application No. 09/712,813, filed on Nov. 13, 2000.
Application No. 09/712,813 is a continuation of application No. PCT/US99/10793, filed on May 14, 1999.
Application No. PCT/US99/10793 is a continuation-in-part of application No. 09/081,385, filed on May 14, 1998, now Pat. No. 6,593,456.
(60) Provisional application No. 60/030,761, filed on Nov. 6, 1996.

(51) Int. Cl. C12Q 1/68; C07H 21/04; C12N 9/64; C12N 15/09

(52) U.S. Cl. 435/6; 435/69.1; 435/226; 435/320.1; 435/325; 536/23.2

(57) ABSTRACT

The biological effects of the cytokine TNF are mediated by binding to receptors on the surface of cells. This disclosure describes new proteins and polynucleotides that promote enzymatic cleavage and release of TNF receptors. Also provided are methods for identifying additional compounds that influence TNF receptor shedding. As the active ingredient in a pharmaceutical composition, the products of this invention increase or decrease TNF signal transduction, thereby alleviating the pathology of disease.

### CROSS-REFERENCE TO RELATED APPLICATIONS

This application claims the priority benefit of U.S. application Ser. No. 09/081,385, filed May 14, 1998, pending. For purposes of prosecution in the U.S., the priority application is hereby incorporated herein by reference in its entirety.

### FIELD OF THE INVENTION

This invention relates generally to the field of signal transduction between cells, via cytokines and their receptors. More specifically, it relates to enzymatic activity that cleaves and releases the receptor for TNF found on the cell surface, and the consequent biological effects. Certain embodiments of this invention are compositions that affect such enzymatic activity, and may be included in medicaments for disease treatment.

### BACKGROUND OF THE INVENTION

Cytokines play a central role in the communication between cells. Secretion of a cytokine from one cell in response to a stimulus can trigger an adjacent cell to undergo an appropriate biological response—such as stimulation, differentiation, or apoptosis. It is hypothesized that important biological events can be influenced not only by affecting cytokine release from the first cell, but also by binding to receptors on the second cell, which mediates the subsequent response. The invention described in this patent application provides new compounds for affecting signal transduction from tumor necrosis factor.

The cytokine known as tumor necrosis factor (TNF or TNF-α) is structurally related to lymphotoxin (LT or TNF-β). They have about 40 percent amino acid sequence homology (Old, Nature 330:602-603, 1987). These cytokines are released by macrophages, monocytes and natural killer cells and play a role in inflammatory and immunological events. The two cytokines cause a broad spectrum of effects both in vitro and in vivo, including: (i) vascular thrombosis and tumor necrosis; (ii) inflammation; (iii) activation of macrophages and neutrophils; (iv) leukocytosis; (v) apoptosis; and (vi) shock. TNF has been associated with a variety of disease states including various forms of cancer, arthritis, psoriasis, endotoxic shock, sepsis, autoimmune diseases, infections, obesity, and cachexia. TNF appears to play a role in the three factors contributing to body weight control: intake, expenditure, and storage of energy (Rothwell, Int. J. Obesity 17:S98-S101, 1993). In septicemia, increased endotoxin concentrations appear to raise TNF levels (Beutler et al. Science 229:869-871, 1985).

Attempts have been made to alter the course of a disease by treating the patient with TNF inhibitors, with varying degrees of success. For example, the TNF inhibitor dexanabinol provided protection against TNF mediated effects following traumatic brain injury (Shohami et al. J. Neuroimmun. 72:169-77, 1997). Some improvement in Crohn's disease was afforded by treatment with anti-TNF antibodies (Neurath et al., Eur. J. Immun. 27:1743-50, 1997).

Human TNF and LT mediate their biological activities by binding specifically to two distinct glycoprotein plasma membrane receptors (55 kDa and 75 kDa in size, known as p55 and p75 TNF-R, respectively). The two receptors share 28 percent amino acid sequence homology in their extracellular domains, which are composed of four repeating cysteine-rich regions (Tartaglia and Goeddel, Immunol Today 13:151-153, 1992). However, the receptors lack significant sequence homology in their intracellular domains, and mediate different intracellular responses to receptor activation. In accordance with the different activities of TNF and LT, most human cells express low levels of both TNF receptors: about 2,000 to 10,000 receptors per cell (Brockhaus et al., Proc. Natl. Acad. Sci. USA 87:3127-3131, 1990).

Expression of TNF receptors on both lymphoid and non-lymphoid cells can be influenced experimentally by many different agents, such as bacterial lipopolysaccharide (LPS), phorbol myristate acetate (PMA; a protein kinase C activator), interleukin-1 (IL-1), interferon-gamma (IFN-γ) and IL-2 (Gatanaga et al. Cell Immunol. 138:1-10, 1991; Yui et al. Placenta 15:819-835, 1994). It has been shown that complexes of human TNF bound to its receptor are internalized from the cell membrane, and then the receptor is either degraded or recycled (Armitage, Curr. Opin. Immunol. 6:407-413, 1994). It has been proposed that TNF receptor activity can be modulated using peptides that bind intracellularly to the receptor, or which bind to the ligand binding site, or that affect receptor shedding. See for example patent publications WO 95/31544, WO 95/33051, WO 96/01642, and EP 568 925.

TNF binding proteins (TNF-BP) have been identified at elevated levels in the serum and urine of febrile patients, patients with renal failure, and cancer patients, and even certain healthy individuals. Human brain and ovarian tumors produced high serum levels of TNF-BP These molecules have been purified, characterized, and cloned (Gatanaga et al., Lymphokine Res. 9:225-229, 1990a; Gatanaga et al., Proc. Natl. Acad. Sci USA 87:8781-8784, 1990b). Human TNF-BP consists of 30 kDa and 40 kDa proteins which are identical to the N-terminal extracellular domains of p55 and p75 TNF receptors, respectively (U.S. Pat. No. 5,395,760; EP 418,014). Such proteins have been suggested for use in treating endotoxic shock. Mohler et al. J. Immunol. 151:1548-1561, 1993.

There are several mechanisms possible for the production of secreted proteins resembling membrane bound receptors. One involves translation from alternatively spliced mRNAs lacking transmembrane and cytoplasmic regions. Another involves proteolytic cleavage of the intact membrane receptors, followed by shedding of the cleaved receptor from the cell. The soluble form of p55 and p75 TNF-R do not appear to be generated from mRNA splicing, since only full length receptor mRNA has been detected in human cells in vitro (Gatanaga et al., 1991). Carboxyl-terminal sequencing and mutation studies on human p55 TNF-R indicates that a cleavage site may exist between residues Asn 172 and Val 173 (Gullberg et al. Eur. J. Cell. Biol. 58:307-312, 1992).

There are reports that a specific metalloprotease inhibitor, TNF-α protease inhibitor (TAPI) blocks the shedding of soluble p75 and p55 TNF-R (Crowe et al. J. Exp. Med. 181:1205-1210, 1995; Mullberg et al. J. Immunol. 155:5198-5205, 1995). The processing of pro-TNF on the cell membrane to release the TNF ligand appears to be dependent on a matrix metalloprotease like enzyme (Gearing et al. Nature 370:555-557, 1994). This is a family of structurally related matrix-degrading enzymes that play a major role in tissue remodeling and repair associated with development and inflammation (Birkedal-Hansen et al. Crit. Rev. Oral Biol. Med. 4:197-250, 1993). The enzymes have Zn²⁺ in their catalytic domains, and Ca²⁺ stabilizes their tertiary structure significantly.

In European patent application EP 657536A1, Wallach et al. suggest that it would be possible to obtain an enzyme that cleaves the 55,000 kDa TNF receptor by finding a mutated form of the receptor that is not cleaved by the enzyme, but still binds to it. The only proposed source for the enzyme is a detergent extract of membranes for cells that appear to have the protease activity. If it were possible to obtain an enzyme according to this scheme, then the enzyme would presumably comprise a membrane spanning region. The patent application does not describe any protease that was actually obtained.

In a previous patent application in the present series (International Patent Publication WO 9820140), methods are described for obtaining an isolated enzyme that cleaves both the p55 and p75 TNF-R from cell surfaces. A convenient source is the culture medium of cells that have been stimulated with phorbol myristate acetate (PMA). The enzyme activity was given the name TRRE (TNF receptor releasing enzyme). In other studies, TRRE was released immediately upon PMA stimulation, indicating that it is presynthesized in an inactive form to be rapidly converted to the active form upon stimulation. Evidence for direct cleavage of TNF-R is that the shedding begins very quickly (˜5 min) with maximal shedding within 30 min. TRRE is specific for the TNF-R, and does not cleave IL-1 receptors, CD30, ICAM-1 or CD11b. TRRE activity is enhanced by adding Ca⁺⁺ or Zn⁺⁺, and inhibited by EDTA and phenantroline.

Given the involvement of TNF in a variety of pathological conditions, it is desirable to obtain a variety of factors that would allow receptor shedding to be modulated, thereby controlling the signal transduction from TNF at a disease site.

### SUMMARY OF THE INVENTION

This disclosure provides new compounds that promote enzymatic cleavage and release of TNF receptors from the cell surface. Nine new DNA clones have been selected after repeat screening in an assay that tests the ability to enhance receptor release. The polynucleotide sequences of this invention and the proteins encoded by them have potential as diagnostic aids, and therapeutic compounds that can be used to adjust TNF signal transduction in a beneficial way.

One embodiment of the invention is an isolated polynucleotide comprising a nucleotide sequence with the following properties: a) the sequence is expressed at the mRNA level in Jurkat T cells; b) when COS-1 cells expressing TNF-receptor are genetically transformed to express the sequence, the cells have increased enzymatic activity for cleaving and releasing the receptor. If a polynucleotide sequence is expressed in Jurkat cells, then it can be found in the Jurkat cell expression library deposited with the ATCC (Accession No. TIB-152). It is recognized that the polynucleotide can be obtained from other cell lines, or produced by recombinant techniques.

Included are polynucleotides in which the nucleotide sequence is contained in any of SEQ. ID NOS:1-10. Also embodied are polynucleotides comprising at least 30 and preferably more consecutive nucleotides in said nucleotide sequence, or at least 50 consecutive nucleotides that are homologous to said sequence at a significant level, preferably at the 90% level or more. Also included antisense and ribozyme polynucleotides that inhibit the expression of a TRRE modulator.

Another embodiment of the invention is isolated polypeptides comprising an amino acid sequence encoded by a polynucleotide of this invention. Non-limiting examples are sequences shown in SEQ. ID NOS: 147-158. Fragments and fusion proteins are included in this invention, and preferably comprise at least 10 consecutive residues encoded by a polynucleotide of this invention, or at least 15 consecutive amino acids that are homologous at a significant level, preferably at least 80%. Preferred polypeptides promote cleavage and release of TNF receptors from the cell surface, especially COS-1 cells genetically transformed to express TNF receptor. The polypeptides may or may not have a membrane spanning domain, and may optionally be produced by a process that involves secretion from a cell. Included are species homologs with the desired activity, and artificial mutants with additional beneficial properties.

Another embodiment of this invention is an antibody specific for a polypeptide of this invention. Preferred are antibodies that bind a TRRE modulator protein, but not other substances found in human tissue samples in comparable amounts.

Another embodiment of the invention is an assay method of determining altered TRRE activity in a cell or tissue sample, using a polynucleotide or antibody of this invention to detect the presence or absence of the corresponding TRRE modulator. The assay method can optionally be used for the diagnosis or evaluation of a clinical condition relating to abnormal TNF levels or TNF signal transduction.

Another embodiment of the invention is a method for increasing or decreasing signal transduction from a cytokine into a cell (including but not limited to TNF), comprising contacting the cell with a polynucleotide, polypeptide, or antibody of this invention.

A further embodiment of the invention is a method for screening polynucleotides for an ability to modulate TRRE activity. The method involves providing cells that express both TRRE and the TNF-receptor; genetically altering the cells with the polynucleotides to be screened; cloning the cells; and identifying clones with the desired activity.

Yet another embodiment of the invention is a method for screening substances for an ability to affect TRRE activity. This typically involves incubating cells expressing TNF receptor with a TRRE modulator of this invention in the presence or absence of the test substance; and measuring the effect on shedding of the TNF receptor.

The products of this invention can be used in the preparation of a medicament for treatment of the human or animal body. The medicament contains a clinically effective amount for treatment of a disease such as heart failure, cachexia, inflammation, endotoxic shock, arthritis, multiple sclerosis, sepsis, and cancer. These compositions can be used for administration to a subject suspected of having or being at risk for the disease, optionally in combination with other forms of treatment appropriate for their condition.

### BRIEF DESCRIPTION OF THE DRAWINGS

FIG. 1 is a schematic representation of plasmid pCDTR2. This plasmid expresses p75 TNF-R, the ˜75 kDa form of the TNF receptor. PCMV stands for cytomegalovirus; BGHPA stands for bovine growth hormone polyadenylation signal.

FIG. 2 is a line depicting the levels of p75 TNF-R detected on COS-1 cells genetically altered to express the receptor. Results from the transformed cells, designated C75R (●, upward swooping line) is compared with that from the parental COS-1 cells (▪, baseline). The receptor number was calculated by Scatchard analysis (inset).

FIG. 3 is a survival graph, showing that TRRE decreases mortality in mice challenged with lipopolysaccharide (LPS) to induce septic peritonitis. (♦) LPS alone; (▪) LPS plus control buffer; (●) LPS plus TRRE (2,000 U); (▴) LPS plus TRRE (4,000 U).

FIG. 4 is a half-tone reproduction of a bar graph, showing the effect of 9 new clones on TRRE activity on C75R cells (COS-1 cells transfected to express the TNF-receptor. Each of the 9 clones increases TRRE activity by over 2-fold.

FIG. 5 is a survival graph, showing the ability of 4 new expressed to save mice challenged with LPS. (♦) saline; (▪) BSA; (Δ) Mey-3 (100 μg); (X) Mey-3 (10 μg); (*) Mey-5 (10 μg); (●) Mey-8 (10 μg).

### DETAILED DESCRIPTION OF THE INVENTION

It has been discovered that certain cells involved in the TNF transduction pathway express enzymatic activity that causes TNF receptors to be shed from the cell surface. Enzymatic activity for cleaving and releasing TNF receptors has been given the designation TRRE. Phorbol myristate acetate induces release of TRRE from cells into the culture medium. An exemplary TRRE protein had been purified from the supernatant of TNF-1 cells (Example 2). The protease bears certain hallmarks of the metalloprotease family, and is released rapidly from the cell upon activation.

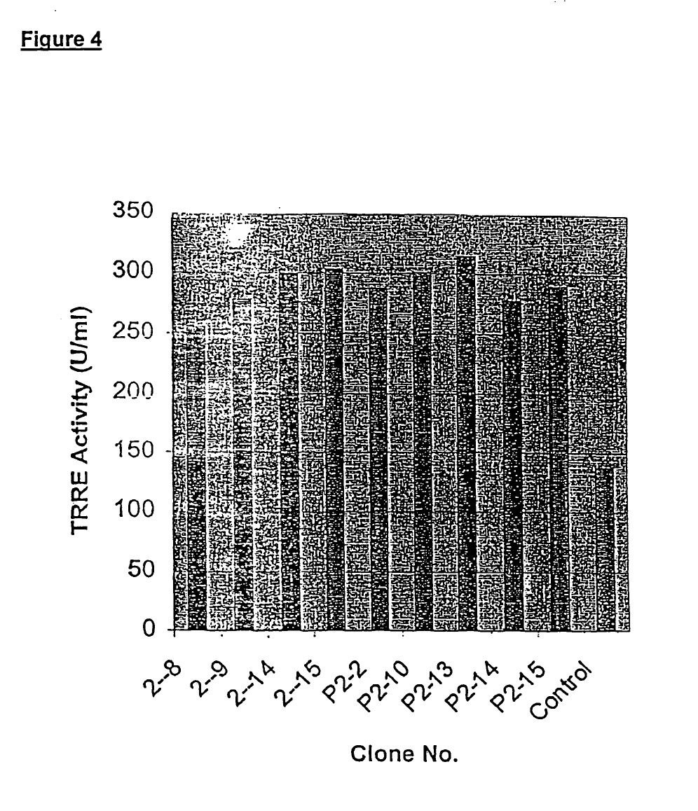

In order to elucidate the nature of this protein, functional cloning was performed. Jurkat cells were selected as being a good source of TRRE. The cDNA from a Jurkat library was expressed, and cell supernatant was tested for an ability to release TNF receptors from cell surfaces. Cloning and testing of the expression product was conducted through several cycles, and nine clones were obtained that more than doubled TRRE activity in the assay (FIG. 4). At the DNA level, all 9 clones had different sequences.

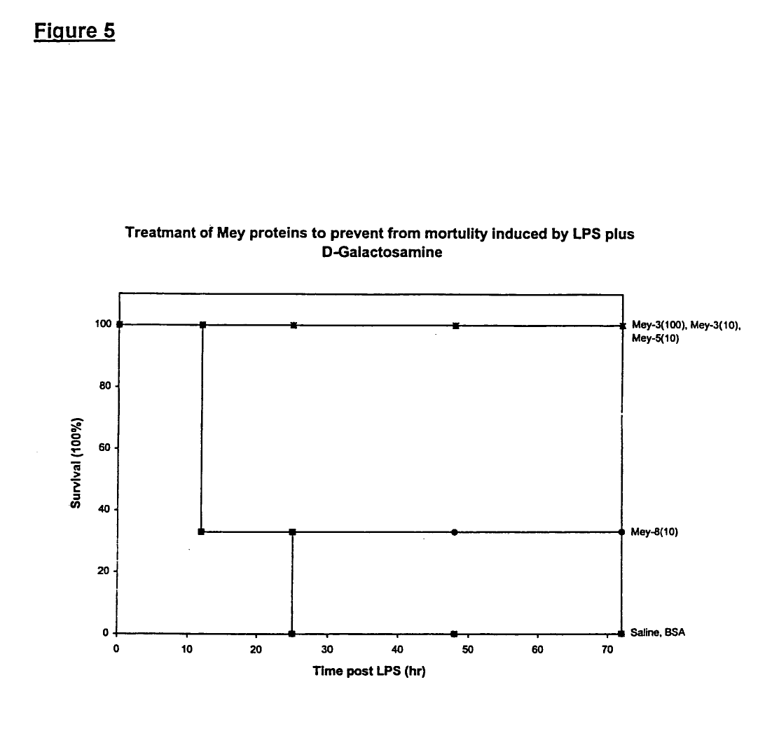

Protein expression products from the clones have been tested in a lipopolysaccharide animal model for sepsis. Protein from three different clones successfully rescued animals from a lethal dose of LPS (FIG. 5). This points to an important role for these molecules in the management of pathological conditions mediated by TNF.

The number of new TRRE promoting clones obtained from the expression library was surprising. The substrate specificity of the TRRE isolated in Example 2 distinguishes the 75 kDa and 55 kDa TNF receptors from other cytokine receptors and cell surface proteins. There was little reason beforehand to suspect that cells might have nine different proteases for the TNF receptor. It is possible that one of the clones encodes the TRRE isolated in Example 2, or a related protein. It is possible that some of the other clones have proteolytic activity to cleave TNF receptors at the same site, or at another site that causes release of the soluble form from the cell. It is a hypothesis of this disclosure that some of the clones may not have proteolytic activity themselves, but play a role in promoting TRRE activity in a secondary fashion.

This possibility is consistent with the observations made, because there is an endogenous level of TRRE activity in the cells used in the assay. The cleavage assay involves monitoring TNF receptor release from C75 cells, which are COS-1 cells genetically altered to express p75 TNF-R. The standard assay is conducted by contacting the transformed cells with a fluid believed to contain TRRE. The level of endogenous TRRE activity is evident from the rate of spontaneous release of the receptor even when no exogenous TRRE is added (about 200 units). Accordingly, accessory proteins that promote TRRE activity would increase the activity measured in the assay. Many mechanisms of promotion are possible, including proteins that activate a zymogen form of TRRE, proteins that free TRRE from other cell surface components, or proteins that stimulate secretion of TRRE from inside the cell. It is not necessary to understand the mechanism in order to use the products of this invention in most of the embodiments described.

It is anticipated that several of the clones will have activity not just for promoting TNF receptor cleavage, but also having an effect on other surface proteins. To the extent that cleavage sequences or accessory proteins are shared between different receptors, certain clones would promote phenotypic change (such as receptor release) for the family of related substrates.

This disclosure provides polypeptides that promote TRRE activity, polynucleotides that encode such polypeptides, and antibodies that bind such peptides. The binding of TNF to its receptor mediates a number of biological effects. Cleavage of the TNF-receptor by TRRE diminishes signal transduction by TRRE. Potentiators of TRRE activity have the same effect. Thus, the products of this invention can be used to modulate signal transduction by cytokines, which is of considerable importance in the management of disease conditions that are affected by cytokine action. The products of this invention can also be used in diagnostic methods, to determine when signal transduction is being inappropriately affected by abnormal TRRE activity. The assay systems described in this disclosure provide a method for screening additional compounds that can influence TRRE activity, and thus the signal transduction from TNF.

Based on the summary of the invention, and guided by the illustrations in the example section, one skilled in the art will readily know what techniques to employ in the practice of the invention. The following detailed description is provided for the additional convenience of the reader.

Definitions and Basic Techniques

As used in this disclosure, “TRRE activity” refers to the ability of a composition to cleave and release TNF receptors from the surface of cells expressing them. A preferred assay is cleavage from transfected COS-1 cells, as described in Example 1. However, TRRE activity can be measured on any cells that bear TNF receptors of the 55 kDa or 75 kDa size. Other features of the TRRE enzyme obtained from PMA induction of THP-1 cells (exemplified in Example 2) need not be a property of the TRRE activity measured in the assay.

Unit activity of TRRE is defined as 1 pg of soluble p75 TNF-R released from cell surface in a standard assay, after correction for spontaneous release. The measurement of TRRE activity is explained further in Example 1.

A “TRRE modulator” is a compound that has the property of either increasing or decreasing TRRE activity for processing TNF on the surface of cells. Those that increase TRRE activity may be referred to as TRRE promoters, and those that decrease TRRE activity may be referred to as TRRE inhibitors. TRRE promoters include compounds that have proteolytic activity for TNF-R, and compounds that augment the activity of TNF-R proteases. The nine polynucleotide clones described in Example 5, and their protein products, are exemplary TRRE promoters. Inhibitors of TRRE activity can be obtained using the screening assays described below.

The term “polynucleotide” refers to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, (mRNA), ribozymes, cDNA, recombinant polynucleotides, branched polynucleotides, plasmids, vectors, nucleic acid probes, and primers. A polynucleotide may comprise modified nucleotides, such as methylated nucleotides and nucleotide analogs. If present, modifications to the nucleotide structure may be imparted before or after assembly of the polymer. The term polynucleotide refers interchangeably to double- and single-stranded molecules. Unless otherwise specified or required, any embodiment of the invention described herein that is a polynucleotide encompasses both the double-stranded form, and each of two complementary single-stranded forms known or predicted to make up the double-stranded form.

“Hybridization” refers to a reaction in which one or more polynucleotides react to form a complex that is stabilized via hydrogen bonding between the bases of the nucleotide residues. Hybridization reactions can be performed under conditions of different “stringency”. Relevant conditions include temperature, ionic strength, and the presence of additional solutes in the reaction mixture such as formamide. Conditions of increasing stringency are 30° C. in 10×SSC (0.15M NaC1, 15 mM citrate buffer); 40° C. in 6×SSC; 50° C. in 6.×SSC 60° C. in 6×SSC, or at about 40° C. in 0.5×SSC, or at about 30° C. in 6.×. SSC containing 50% formamide. SDS and a source of fragmented DNA (such as salmon sperm) are typically also present during hybridization. Higher stringency requires higher minimum complementarity between hybridizing elements for a stable hybridization complex to form. See “Molecular Cloning: A Laboratory Manual”, Second Edition (Sambrook, Fritsch & Maniatis, 1989).

It is understood that purine and pyrimidine nitrogenous bases with similar structures can be functionally equivalent in terms of Watson-Crick base-pairing; and the inter-substitution of like nitrogenous bases, particularly uracil and thymine, or the modification of nitrogenous bases, such as by methylation, does not constitute a material substitution.

The percentage of sequence identity for polynucleotides or polypeptides is calculated by aligning the sequences being compared, and then counting the number of shared residues at each aligned position. No penalty is imposed for the presence of insertions or deletions, but are permitted only where required to accommodate an obviously increased number of amino acid residues in one of the sequences being aligned. When one of the sequences being compared is indicated as being “consecutive”, then no gaps are permitted in that sequence during the comparison. The percentage identity is given in terms of residues in the test sequence that are identical to residues in the comparison or reference sequence.

As used herein, “expression” of a polynucleotide refers to the production of an RNA transcript. Subsequent translation into protein or other effector compounds may also occur, but is not required unless specified.

“Genetic alteration” refers to a process wherein a genetic element is introduced into a cell other than by mitosis or meiosis. The element may be heterologous to the cell, or it may be an additional copy or improved version of an element already present in the cell. Genetic alternation may be effected, for example, by transducing a cell with a recombinant plasmid or other polynucleotide through any process known in the art, such as electroporation, calcium phosphate precipitation, or contacting with a polynucleotide-liposome complex. Genetic alteration may also be effected, for example, by transduction or infection with a DNA or RNA virus or viral vector. It is preferable that the genetic alteration is inheritable by progeny of the cell, but this is not generally required unless specified.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component.

A “fusion polypeptide” is a polypeptide comprising regions in a different position in the sequence than occurs in nature. The regions can normally exist in separate proteins and are brought together in the fusion polypeptide; they can normally exist in the same protein but are placed in a new arrangement in the fusion polypeptide; or they can be synthetically arranged. A “functionally equivalent fragment” of a polypeptide varies from the native sequence by addition, deletion, or substitution of amino acid residues, or any combination thereof, while preserving a functional property of the fragment relevant to the context in which it is being used. Fusion peptides and functionally equivalent fragments are included in the definition of polypeptides used in this disclosure.

It is understood that the folding and the biological function of proteins can accommodate insertions, deletions, and substitutions in the amino acid sequence. Some amino acid substitutions are more easily tolerated. For example, substitution of an amino acid with hydrophobic side chains, aromatic side chains, polar side chains, side chains with a positive or negative charge, or side chains comprising two or fewer carbon atoms, by another amino acid with a side chain of like properties can occur without disturbing the essential identity of the two sequences. Methods for determining homologous regions and scoring the degree of homology are described in Altschul et al. Bull. Math. Bio. 48:603-616, 1986; and Henikoff et al. Proc. Natl. Acad. Sci. USA 89:10915-10919, 1992. Substitutions that preserve the functionality of the polypeptide, or confer a new and beneficial property (such as enhanced activity, stability, or decreased immunogenicity) are especially preferred.

An “antibody” (interchangeably used in plural form) is an immunoglobulin molecule capable of specific binding to a target, such as a polypeptide, through at least one antigen recognition site, located in the variable region of the immunoglobulin molecule. As used herein, the term encompasses not only intact antibodies, but also antibody equivalents that include at least one antigen combining site of the desired specificity. These include but are not limited to enzymatic or recombinantly produced fragments antibody, fusion proteins, humanized antibodies, single chain variable regions, diabodies, and antibody chains that undergo antigen-induced assembly.

An “isolated” polynucleotide, polypeptide, protein, antibody, or other substance refers to a preparation of the substance devoid of at least some of the other components that may also be present where the substance or a similar substance naturally occurs or is initially obtained from. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this invention are increasingly more preferred. Thus, for example, a 2-fold enrichment is preferred, 10-fold enrichment is more preferred, 100-fold enrichment is more preferred, 1000-fold enrichment is even more preferred. A substance can also be provided in an isolated state by a process of artificial assembly, such as by chemical synthesis or recombinant expression.

A “host cell” is a cell which has been genetically altered, or is capable of being transformed, by administration of an exogenous polynucleotide.

The term “clinical sample” encompasses a variety of sample types obtained from a subject and useful in an in vitro procedure, such as a diagnostic test. The definition encompasses solid tissue samples obtained as a surgical removal, a pathology specimen, or a biopsy specimen, cells obtained from a clinical subject or their progeny obtained from culture, liquid samples such as blood, serum, plasma, spinal fluid, and urine, and any fractions or extracts of such samples that contain a potential indication of the disease.

Unless otherwise indicated, the practice of the invention will employ conventional techniques of molecular biology, microbiology, recombinant DNA, and immunology, within the skill of the art. Such techniques are explained in the standard literature, such as: “Molecular Cloning: A Laboratory Manual”, Second Edition (Sambrook, Fritsch & Maniatis, 1989), “Oligonucleotide Synthesis” (M. J. Gait, ed., 1984), “Animal Cell Culture” (R. I. Freshney, ed., 1987); the series “Methods in Enzymology” (Academic Press, Inc.); “Handbook of Experimental Immunology” (D. M. Weir & C. C. Blackwell, Eds.), “Gene Transfer Vectors for Mammalian Cells” (J. M. Miller & M. P. Calos, eds., 1987), “Current Protocols in Molecular Biology” (F. M. Ausubel et al., eds., 1987); and “Current Protocols in Immunology” (J. E. Coligan et al., eds., 1991). The reader may also choose to refer to a previous patent application relating to TRRE, International Patent Application WO 98020140.

For purposes of prosecution in the U.S., and in other jurisdictions where allowed, all patents, patent applications, articles and publications indicated anywhere in this disclosure are hereby incorporated herein by reference in their entirety.

Polynucleotides

Polynucleotides of this invention can be prepared by any suitable technique in the art. Using the data provided in this disclosure, sequences of less than ˜50 base pairs are conveniently prepared by chemical synthesis, either through a commercial service or by a known synthetic method, such as the triester method or the phosphite method. A preferred method is solid phase synthesis using mononucleoside phosphoramidite coupling units (Hirose et al., Tetra. Lett. 19:2449-2452, 1978; U.S. Pat. No. 4,415,732).

For use in antisense therapy, polynucleotides can be prepared by chemistry that produce more stable in pharmaceutical preparations. Non-limiting examples include thiol-derivatized nucleosides (U.S. Pat. No. 5,578,718), and oligonucleotides with modified backbones (U.S. Pat. Nos. 5,541,307 and 5,378,825).

Polynucleotides of this invention can also be obtained by PCR amplification of a template with the desired sequence. Oligonucleotide primers spanning the desired sequence are annealed to the template, elongated by a DNA polymerase, and then melted at higher temperature so that the template and elongated oligonucleotides dissociate. The cycle is repeated until the desired amount of amplified polynucleotide is obtained (U.S. Pat. Nos. 4,683,195 and 4,683,202). Suitable templates include the Jurkat T cell library and other human or animal expression libraries that contain TRRE modulator encoding sequences. The Jurkat T cell library is available from the American Type Culture Collection, 10801 University Blvd., Manassas Va. 20110, U.S.A. (ATCC #TIB-152). Mutations and other adaptations can be performed during amplification by designing suitable primers, or can be incorporated afterwards by genetic splicing.

Production scale amounts of large polynucleotides are most conveniently obtained by inserting the desired sequence into a suitable cloning vector and reproducing the clone. Techniques for nucleotide cloning are given in Sambrook, Fritsch & Maniatis (supra) and in U.S. Pat. No. 5,552,524. Exemplary cloning and expression methods are illustrated in Example 6.

Preferred polynucleotide sequences are 50%, 70%, 80%, 90%, or 100% identical to one of the sequences exemplified in this disclosure; in order if increasing preference. The length of consecutive residues in the identical or homologous sequence compared with the exemplary sequence can be about 15, 30, 50, 75, 100, 200 or 500 residues in order of increasing preference, up to the length of the entire clone. Nucleotide changes that cause a conservative substitution or retain the function of the encoded polypeptide (in terms of hybridization properties or what is encoded) are especially preferred substitutions.

The polynucleotides of this can be used to measure altered TRRE activity in a cell or tissue sample. This involves contacting the sample with the polynucleotide under conditions that permit the polynucleotide to hybridize specifically with nucleic acid that encodes a modulator of TRRE activity, if present in the sample, and determining polynucleotide that has hybridized as a result of step a). Specificity of the test can be provided in one of several ways. One method involves the use of a specific probe—a polynucleotide of this invention with a sequence long enough and of sufficient identity to the sequence being detected, so that it binds the target and not other nucleic acid that might be present in the sample. The probe is typically labeled (either directly or through a secondary reagent) so that it can be subsequently detected. Suitable labels include ³²p and ³³P, chemiluminescent and fluorescent reagents. After the hybridization reaction, unreacted probe is washed away so that the amount of hybridized probe can be determined. Signal can be amplified using branched probes (U.S. Pat. No. 5,124,246). In another method, the polynucleotide is a primer for a PCR reaction. Specificity is provided by the ability of the paired probes to amplify the sequence of interest. After a suitable number of PCR cycles, the amount of amplification product present correlates with the amount of target sequence originally present in the sample.

Such tests are useful both in research, and in the diagnosis or assessment of a disease condition. For example, TNF activity plays a role in eliminating tumor cells (Example 4), and a cancer may evade the elimination process by activating TRRE activity in the diseased tissue. Hence, under some conditions, high expression of TRRE modulators may correlate with progression of cancer. Diagnostic tests are also of use in monitoring therapy, such as when gene therapy is performed to increase TRRE activity.

Polynucleotides of this invention can also be used for production of polypeptides and the preparation of medicaments, as explained below.

Polypeptides

Short polypeptides of this invention can be prepared by solid-phase chemical synthesis. The principles of solid phase chemical synthesis can be found in Dugas & Penney, Bioorganic Chemistry, Springer-Verlag NY pp 54-92 (1981), and U.S. Pat. No. 4,493,795. Automated solid-phase peptide synthesis can be performed using devices such as a PE-Applied Biosystems 430A peptide synthesizer (commercially available from Applied Biosystems, Foster City Calif.).

Longer polypeptides are conveniently obtained by expression cloning. A polynucleotide encoding the desired polypeptide is operably linked to control elements for transcription and translation, and then transfected into a suitable host cell. Expression may be effected in procaryotes such as E. coli (ATCC Accession No. 31446 or 27325), eukaryotic microorganisms such as the yeast Saccharomyces cerevisiae, or higher eukaryotes, such as insect or mammalian cells. A number of expression systems are described in U.S. Pat. No. 5,552,524. Expression cloning is available from such commercial services as Lark Technologies, Houston Tex. The production of protein from 4 exemplary clones of this invention in insect cells is illustrated in Example 6. The protein is purified from the producing host cell by standard methods in protein chemistry, such as affinity chromatography and HPLC. Expression products are optionally produced with a sequence tag to facilitate affinity purification, which can subsequently be removed.

Preferred sequences are 40%, 60%, 80%, 90%, or 100% identical to one of the sequences exemplified in this disclosure; in order if increasing preference. The length of the identical or homologous sequence compared with the native human polynucleotide can be about 7, 10, 15, 20, 30, 50 or 100 residues in order of increasing preference, up to the length of the entire encoding region.

Polypeptides can be tested for an ability to modulate TRRE in a TNF-R cleavage assay. The polypeptide is contacted with the receptor (preferably expressed on the surface of a cell, such as a C75 cell), and the ability of the polypeptide to increase or decrease receptor cleavage and release is determined. Cleavage of TNF-R by exemplary polypeptides of this invention is illustrated in Example 7.

Polypeptides of this invention can be used as immunogens for raising antibody. Large proteins will raise a cocktail of antibodies, while short peptide fragments will raise antibodies against small region of the intact protein. Antibody clones can be mapped for protein binding site by producing short overlapping peptides of about 10 amino acids in length. Overlapping peptides can be prepared on a nylon membrane support by standard F-Moc chemistry, using a SPOTS™ kit from Genosys according to manufacturer's directions.

Polypeptides of this invention can also be used to affect TNF signal transduction, as explained below.

Antibodies

Polyclonal antibodies can be prepared by injecting a vertebrate with a polypeptide of this invention in an immunogenic form. Immunogenicity of a polypeptide can be enhanced by linking to a carrier such as KLH, or combining with an adjuvant, such as Freund's adjuvant. Typically, a priming injection is followed by a booster injection is after about 4 weeks, and antiserum is harvested a week later. Unwanted activity cross-reacting with other antigens, if present, can be removed, for example, by running the preparation over adsorbants made of those antigens attached to a solid phase, and collecting the unbound fraction. If desired, the specific antibody activity can be further purified by a combination of techniques, which may include protein, A chromatography, ammonium sulfate precipitation, ion exchange chromatography, HPLC, and immunoaffinity chromatography using the immunizing polypeptide coupled to a solid support. Antibody fragments and other derivatives can be prepared by standard immunochemical methods, such as subjecting the antibody to cleavage with enzymes such as papain or pepsin.

Production of monoclonal antibodies is described in such standard references as Harrow & Lane (1988), U.S. Pat. Nos. 4,491,632, 4,472,500 and 4,444,887, and Methods in Enzymology 73B:3 (1981). Briefly, a mammal is immunized, and antibody-producing cells (usually splenocytes) are harvested. Cells are immortalized by fusion with a non-producing myeloma, transfecting with Epstein Barr Virus, or transforming with oncogenic DNA. The treated cells are cloned and cultured, and the clones are selected that produce antibody of the desired specificity.

Other methods of obtaining specific antibody molecules (optimally in the form of single-chain variable regions) involve contacting a library of immunocompetent cells or viral particles with the target antigen, and growing out positively selected clones. Immunocompetent phage can be constructed to express immunoglobulin variable region segments on their surface. See Marks et al., New Eng. J. Med. 335:730, 1996, International Patent Applications WO 9413804, WO 9201047, WO 90 02809, and McGuiness et al., Nature Biotechnol. 14:1449, 1996.

The antibodies of this invention are can be used in immunoassays for TRRE modulators. General techniques of immunoassay can be found in “The Immunoassay Handbook”, Stockton Press NY, 1994; and “Methods of Immunological Analysis”, Weinheim: VCH Verlags gesellschaft mbH, 1993). The antibody is combined with a test sample under conditions where the antibody will bind specifically to any modulator that might be present, but not any other proteins liable to be in the sample. The complex formed can be measured in situ (U.S. Pat. Nos. 4,208,479 and 4,708,929), or by physically separating it from unreacted reagents (U.S. Pat. No. 3,646,346). Separation assays typically involve labeled TRRE reagent (competition assay), or labeled antibody (sandwich assay) to facilitate detection and quantitation of the complex. Suitable labels are radioisotopes such as ¹²⁵I, enzymes such as β-galactosidase, and fluorescent labels such as fluorescein. Antibodies of this invention can also be used to detect TRRE modulators in fixed tissue sections by immunohistology. The antibody is contacted with the tissue, unreacted antibody is washed away, and then bound antibody is detected—typically using a labeled anti-immunoglobulin reagent. Immunohistology will show not only whether the modulator is present, but where it is located in the tissue.

Detection of TRRE modulators is of interest for research purposes, and for clinical use. As indicated earlier, high expression of TRRE modulators may correlate with progression of cancer. Diagnostic tests are also of use in monitoring TRRE modulators that are administered in the course of therapy.

Antibodies of this invention can also be used for preparation of medicaments. Antibodies with therapeutic potential include those that affect TRRE activity—either by promoting clearance of a TRRE modulator, or by blocking its physiological action. Antibodies can be screened for desirable activity according to assays described in the next section.

Screening Assays

This invention provides a number of screening methods for selecting and developing products that modulate TRRE, and thus affect TNF signal transduction.

One screening method is for polynucleotides that have an ability to modulate TRRE activity. To do this screening, cells are obtained that express both TRRE and the TNF receptor. Suitable cell lines can be constructed from any cell that expresses a level of functional TRRE activity. These cells are identifiable by testing culture supernatant for an ability to release membrane-bound TNF-R. The level of TRRE expression should be moderate, so that an increase in activity can be detected. The cells can then be genetically altered to express either p55 or p75 TNF-R, illustrated in Example 1. Exemplary is the C75R line: COS-1 cells genetically altered to express the 75 kDa form of the TNF-R. Release of TNF-R from the cell can be measured either by testing residual binding of labeled TNF ligand to the cell, or by immunoassay of the supernatant for released receptor (Example 1).

The screening assay is conducted by contacting the cells expressing TRRE and TNF-R with the polynucleotides to be screened. The effect of the polynucleotide on the enzymatic release of TNF-R from the cell is determined, and polynucleotides with desirable activity (either promoting or inhibiting TRRE activity) are selected. In a variation of this method, cells expressing TRRE activity but not TNF-R (such as untransfected COS-1 cells) are contacted with the test polynucleotide. Then the culture medium is collected, and used to assay for TRRE activity using a second cell expressing TNF-R (such as C75 cells).

This type of screening assay is useful for the selection of polynucleotides from an expression library believed to contain encoding sequences for TRRE modulators. The Jurkat cell expression library (ATCC Accession No. TIB-152) is exemplary. Other cells from which suitable libraries can be constructed are those known to express high levels of TRRE, especially after PMA stimulation, such as THP-1, U-937, HL-60, ME-180, MRC-5, Raji, K-562, and normal human monocytes. The screening involves expressing DNA from the library in the selected cell line being used for screening. Wells with the desired activity are selected, and the DNA is recovered, optionally after replication or cloning of the cells. Repeat cycles of functional screening and selection can lead to identification of new polynucleotide clones that promote or inhibit TRRE activity. This is illustrated below in Example 5. Further experiments can be performed on the selected polynucleotides to determine it modulates TRRE activity inside the cell, or through the action of a protein product. A long open reading frame suggests a role for a protein product, and examination of the amino acid sequence for a signal peptide and a membrane spanning region can help determine whether the protein is secreted from the cell or expressed in the surface membrane.

This type of screening is also useful for further development of the polynucleotides of this invention. For example, expression constructs can be developed that encode functional peptide fragments, fusion proteins, and other variants. The minimum size of polynucleotide sequence that still encodes TRRE modulation activity can be determined by removing part of the sequence and then using the screening assay to determine whether the activity is still present. Mutated and extended sequences can be tested in the same way.

This type of screening assay is also useful for developing compounds that affect TRRE activity by interfering with mRNA that encode a TRRE modulator. Of particular interest are ribozymes and antisense oligonucleotides. Ribozymes are endoribonucleases that catalyze cleavage of RNA at a specific site. They comprise a polynucleotide sequence that is complementary to the cleavage site on the target, and additional sequence that provide the tertiary structure to effect the cleavage. Construction of ribozymes is described in U.S. Pat. Nos. 4,987,071 and 5,591,610. Antisense oligonucleotides that bind mRNA comprise a short sequence complementary to the mRNA (typically 8-25 bases in length). Preferred chemistry for constructing antisense oligonucleotides is outlined in an earlier section. Specificity is provided both by the complementary sequence, and by features of the chemical structure. Antisense molecules that inhibit expression of cell surface receptors are described in U.S. Pat. Nos. 5,135,917 and 5,789,573. Screening involves contacting the cell expressing TRRE activity and TNF-R with the compound and determining the effect on receptor release. Ribozymes and antisense molecules effective in altering expression of a TRRE promoter would decrease TNF-R release. Ribozymes and antisense molecules effective in altering expression of a TRRE inhibitor would increase TNF-R release.

Another screening method described in this disclosure is for testing the ability of polypeptides to modulate TRRE activity (Example 7). Cells expressing both TNF-R and a moderate level of TRRE activity are contacted with the test polypeptides, and the rate of receptor release is compared with the rate of spontaneous release. An increased rate of release indicates that the polypeptide is a TRRE promoter, while a decreased rate indicates that the polypeptide is a TRRE inhibitor. This assay can be used to test the activity of new polypeptides, and develop variants of polypeptides already known to modulate TRRE. The minimum size of polypeptide sequence that still encodes TRRE modulation activity can be determined by making a smaller fragment of the polypeptide and then using the screening assay to determine whether the activity is still present. Mutated and extended sequences can be tested in the same way.

Another screening method embodied in this invention is a method for screening substances that interfere with the action of a TRRE modulator at the protein level. The method involves incubating cells expressing TNF receptor (such as C75R cells) with a polypeptide of this invention having TNF promoting activity. There are two options for supplying the TRRE modulator in this assay. In one option, the polypeptide is added to the medium of the cells as a reagent, along with the substance to be tested. In another option, the cells are genetically altered to express the TRRE modulator at a high level, and the assay requires only that the test substance be contacted with the cells. This option allows for high throughput screening of a number of test compounds.

Either way, the rate of receptor release is compared in the presence and absence of the test substance, to identify compounds that enhance or diminish TRRE activity. Parallel experiments should be conducted in which the activity of the substance on receptor shedding is tested in the absence of added polypeptide (using cells that don't express the polypeptide). This will determine whether the activity of the test substance occurs via an effect on the TRRE promoter being added, or through some other mechanism.

This type of screening assay is useful for identifying antibodies that affect the activity of a TRRE modulator. Antibodies are raised against a TRRE modulator as described in the previous section. If the antibody decreases TRRE activity in the screening assay, then it has therapeutic potential to lower TRRE activity in vivo. Screening of monoclonal antibodies using this assay can also help identify binding or catalytic sites in the polypeptide.

This type of screening assay is also useful for high throughput screening of small molecule compounds that have the ability to affect the level of TNF receptors on a cell, by way of its influence on a TRRE modulator. Small molecule compounds that have the desired activity are often preferred for pharmaceutical compositions, because they are often more stable and less expensive to produce.

Medicaments and their Use

As described earlier, a utility of certain products embodied in this invention is to affect signal transduction from cytokines (particularly TNF). Products that promote TRRE activity have the effect of decreasing TNF receptors on the surface of cells, which would decrease signal transduction from TNF. Conversely, products that inhibit TRRE activity prevent cleavage of TNF receptors, increasing signal transduction.

The ability to affect TNF signal transduction is of considerable interest in the management of clinical conditions in which TNF signaling contributes to the pathology of the condition. Such conditions include:
Heart failure. IL-1β and TNF are believed to be central mediators for perpetuating the inflammatory process, recruiting and activating inflammatory cells. The inflammation depress cardiac function in congestive heart failure, transplant rejection, myocarditis, sepsis, and burn shock.
Cachexia. The general weight loss and wasting occurring in the course of chronic diseases, such as cancer. TNF is believed to affect appetite, energy expenditure, and metabolic rate.
Crohn's disease. The inflammatory process mediated by TNF leads to thickening of the intestinal wall, ensuing from lymphedema and lymphocytic infiltration.
Endotoxic shock. The shock induced by release of endotoxins from gram-negative bacteria, such as E. coli, involves TNF-mediated inflammation
Arthritis. TNF promotes expression of nitric oxide synthetase, believed to be involved in disease pathogenesis.
   Other conditions of interest are multiple sclerosis, sepsis, inflammation brought on by microbe infection, and diseases that have an autoimmune etiology, such as Type I Diabetes.

Polypeptides of this invention that promote TRRE activity can be administered with the objective of decreasing or normalizing TNF signal transduction. For example, in congestive heart failure or Crohn's disease, the polypeptide is given at regular intervals to lessen the inflammatory sequelae. The treatment is optionally in combination with other agents that affect TNF signal transduction (such as antibodies to TNF or receptor antagonists) or that lessen the extent of inflammation in other ways.

Polynucleotides of this invention can also be used to promote TRRE activity by gene therapy. The encoding sequence is operably linked to control elements for transcription and translation in human cells. It is then provided in a form that will promote entry and expression of the encoding sequence in cells at the disease site. Forms suitable for local injection include naked DNA, polynucleotides packaged with cationic lipids, and polynucleotides in the form of viral vectors (such as adenovirus and AAV constructs). Methods of gene therapy known to the practitioner skilled in the art will include those outlined in U.S. Pat. Nos. 5,399,346, 5,827,703, and 5,866,696.

The ability to affect TNF signal transduction is also of interest where TNF is thought to play a beneficial role in resolving the disease. In particular, TNF plays a beneficial role in the necrotizing of solid tumors. Accordingly, products of this invention can be administered to cancer patients to inhibit TRRE activity, thereby increasing TNF signal transduction and improve the beneficial effect.

Embodiments of the invention that inhibit TRRE activity include antisense polynucleotides. A method of conferring long-standing inhibitory activity is to administer antisense gene therapy. A genetic construct is designed that will express RNA inside the cell which in turn will decrease the transcription of the target gene (U.S. Pat. No. 5,759,829). In humans, a more frequent form of antisense therapy is to administer the effector antisense molecule directly, in the form of a short stable polynucleotide fragment that is complementary to a segment of the target mRNA (U.S. Pat. Nos. 5,135,917 and 5,789,573)—in this case, the transcript that encodes the TRRE modulator. Another embodiment of the invention that inhibits TRRE are ribozymes, constructed as described in an earlier section. The function of ribozymes in inhibiting mRNA translation is described in U.S. Pat. Nos. 4,987,071 and 5,591,610.

Once a product of this invention is found to have suitable TRRE modulation activity in the in vitro assays described in this disclosure, it is preferable to also test its effectiveness in an animal model of a TNF mediated disease process. Example 3 describes an LPS model for sepsis that can be used to test promoters of TRRE activity. Example 4 describes a tumor necrosis model, in which TRRE inhibitors could be tested for an ability to enhance necrotizing activity. Those skilled in the art will know of other animal models suitable for testing effects on TNF signal transduction or inflammation. Other illustrations are the cardiac ischemia reperfusion models of Weyrich et al. (J. Clin. Invest. 91:2620, 1993) and Garcia-Criado et al. (J. Am. Coll. Surg. 181:327, 1995); the pulmonary ischemia reperfusion model of Steinberg et al. (J. Heart Lung Transplant. 13:306, 1994), the lung inflammation model of International Patent Application WO 9635418; the bacterial peritonitis model of Sharar et al. (J. Immunol. 151:4982, 1993), the colitis model of Meenan et al. (Scand. J. Gastroenterol. 31:786, 1996), and the diabetes model of von Herrath et al. (J. Clin. Invest 98:1324, 1996). Models for septic shock are described in Mack et al. J. Surg. Res. 69:399, 1997; and Seljelid et al. Scand. J. Immunol 45:683-7.

For use as an active ingredient in a pharmaceutical preparation, a polypeptide, polynucleotide, or antibody of this invention is generally purified away from other reactive or potentially immunogenic components present in the mixture in which they are prepared. Typically, each active ingredient is provided in at least about 90% homogeneity, and more preferably 95% or 99% homogeneity, as determined by functional assay, chromatography, or SDS polyacrylamide gel electrophoresis. The active ingredient is then compounded into a medicament in accordance with generally accepted procedures for the preparation of pharmaceutical preparations, such as described in Remington's Pharmaceutical Sciences 18th Edition (1990), E. W. Martin ed., Mack Publishing Co., PA. Steps in the compounding of the medicament depend in part on the intended use and mode of administration, and may include sterilizing, mixing with appropriate non-toxic and non-interfering excipients and carriers, dividing into dose units, and enclosing in a delivery device. The medicament will typically be packaged with information about its intended use.

Mode of administration will depend on the nature of the condition being treated. For conditions that are expected to require moderate dosing and that are at well perfused sites (such as cardiac failure), systemic administration is acceptable. For example, the medicament may be formulated for intravenous administration, intramuscular injection, or absorption sublingually or intranasally. Where it is possible to administer the active ingredient locally, this is usually preferred. Local administration will both enhance the concentration of the active ingredient at the disease site, and minimize effects on TNF receptors on other tissues not involved in the disease process. Conditions that lend themselves to administration directly at the disease site include cancer and rheumatoid arthritis. Solid tumors can be injected directly when close to the skin, or when they can be reached by an endoscopic procedure. Active ingredients can also be administered to a tumor site during surgical resection, being implanted in a gelatinous matrix or in a suitable membrane such as Gliadel® (Guilford Sciences). Where direct administration is not possible, the administration may be given through an arteriole leading to the disease site. Alternatively, the pharmaceutical composition may be formulated to enhance accumulation of the active ingredient at the disease site. For example, the active ingredient can be encapsulated in a liposome or other matrix structure that displays an antibody or ligand capable of binding a cell surface protein on the target cell. Suitable targeting agents include antibodies against cancer antigens, ligands for tissue-specific receptors (e.g., serotonin for pulmonary targeting). For compositions that decrease TNF signal transduction, an appropriate targeting molecule may be the TNF ligand, since the target tissue may likely display an unusually high density of the TNF receptor.

Effective amounts of the compositions of the present invention are those that alter TRRE activity by at least about 10%, typically by at least about 25%, more preferably by about 50% or 75%. Where near complete ablation of TRRE activity is desirable, preferred compositions decrease TRRE activity by at least 90%. Where increase of TRRE activity is desirable, preferred compositions increase TRRE activity by at least 2-fold. A minimum effective amount of the active compound will depend on the disease being treated, which of the TRRE modulators is selected for use, and whether the administration will be systemic or local. For systemic administration, an effective amount of activity will generally be an amount of the TRRE modulator that can cause a change in the enzyme activity by 100 to 50,000 Units—typically about 10,000 Units. The mass amount of protein, nucleic acid, or antibody is chosen accordingly, based on the specific activity of the active compound in Units per gram.

The following examples provided as a further guide to the practitioner, and are not intended to limit the invention in any way.

### EXAMPLES

### Example 1

### Assay System for TRRE Activity

This Example illustrates an assay system that measures TRRE activity on the human TNF-R in its native conformation in the cell surface membrane.

Membrane-associated TNF-R was chosen as the substrate, as having microenvironment similar to that of the substrate for TRRE in vivo. Membrane-associated TNF-R also requires more specific activity, which would differentiate less-specific proteases. Cells expressing an elevated level of the p75 form of TNF-R were constructed by cDNA transfection into monkey COS-1 cells which express little TNF-R of either the 75 kDa or 55 kDa size.

The procedure for constructing these cells was as follows: cDNA of human p75.TNF-R was cloned from a λgt10 cDNA library derived from human monocytic U-937 cells (Clontech Laboratories, Palo Alto, Calif.). The first 300 bp on both 5′ and 3′ ends of the cloned fragment was sequenced and compared to the reported cDNA sequence of human p75 TNF-R. The cloned sequence was a 2.3 kb fragment covering positions 58-2380 of the reported p75 TNF-R sequence, which encompasses the full length of the p75 TNF-R-coding sequence from positions 90-1475. The 2.3 kb p75 TNF-R cDNA was then subcloned into the multiple cloning site of the pCDNA3 eukaryotic expression vector. The orientation of the p75 TNF-R cDNA was verified by restriction endonuclease mapping.

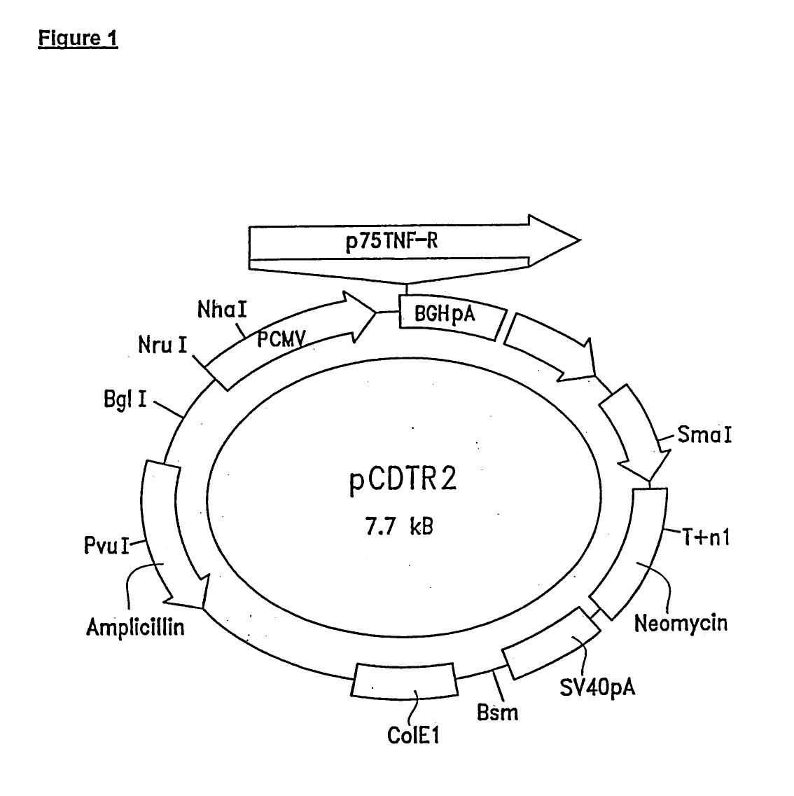

FIG. 1 illustrates the final 7.7 kb construct, pCDTR2. It carries the neomycin-resistance gene for the selection of transfected cells in G418, and the expression of the p75 TNF-R is driven by, the cytomegalovirus promoter. The pCDTR2 was then transfected into monkey kidney COS-1 cells (ATCC CRL-1650) using the calcium phosphate-DNA precipitation method. The selected clone in G418 medium was identified and subcultured. This clone was given the designation C75R.

To determine the level of p75 TNF-R expression on C75R cells, 2×10⁵ cells/well were plated into a 24-well culture plate and incubated for 12 to 16 hours in 5% CO₂ at 37° C. They were then incubated with 2-30 ng ¹²⁵I human recombinant TNF (radiolabeled using the chloramine T method) in the presence or absence of 100-fold excess of unlabeled human TNF at 4° C. for 2 h. After three washes with ice-cold PBS, cells were lysed with 0.1N NaOH and bound radioactivity was determined in a Pharmacia Clinigamma counter (Uppsala, Sweden).

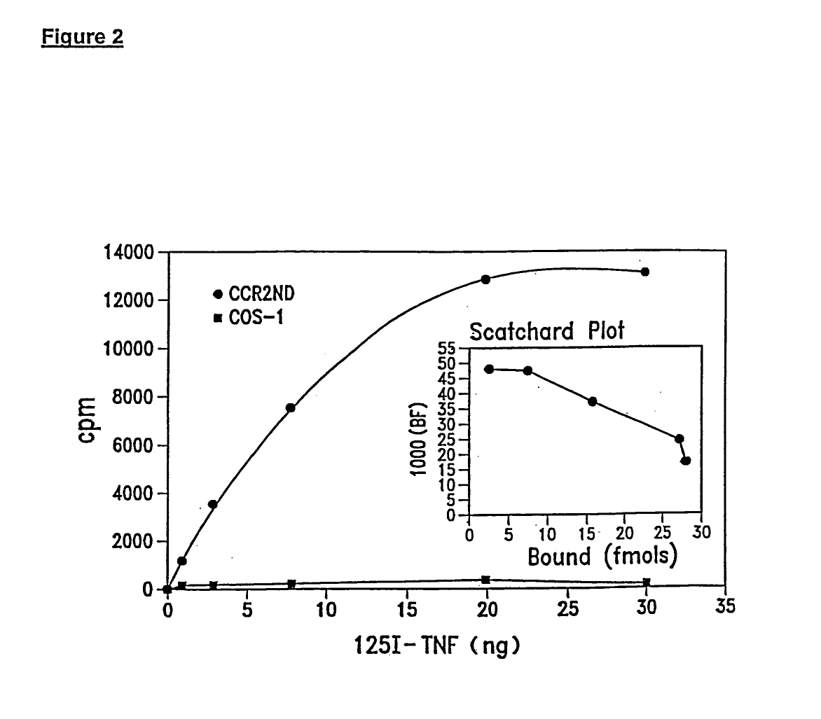

FIG. 2 shows the results obtained. C75R had a very high level of specific binding of radiolabeled ¹²⁵I-TNF, while parental COS-1 cells did not. The number of TNF-R expressed on C75R was determined to be 60,000-70,000 receptors per cell by Scatchard analysis (FIG. 2, inset). The Kd value calculated was 5.6×10⁻¹⁰ M. This Kd value was in close agreement to the values previously reported for native p75 TNF-R.

TRRE was obtained by PHA stimulation of THP-1 cells (WO 9802140). THP-1 cells (ATCC 45503) growing in logarithmic phase were collected and resuspended to 1×10⁶ cells/ml of RPMI-1640 supplemented with 1% FCS and incubated with 10⁻⁶ M PMA for 30 min in 5% CO₂ at 37° C. The cells were collected and washed once with serum-free medium to remove PMA and resuspended in the same volume of RPMI-1640 with 1% FCS. After 2 hours incubation in 5% CO₂ at 37° C., the cell suspension was collected, centrifuged, and the cell-free supernatant was collected as the source of TRRE.

In order to measure the effect of TRRE on membrane-bound TNF-R in the COS-1 cell constructs, the following experiment was performed. C75R cells were seeded at a density of 2×10⁵ cells/well in a 24-well cell culture plate and incubated for 12 to 16 hours at 37° C. in 5% CO₂. The medium in the wells was aspirated, replaced with fresh medium alone or with TRRE medium, and incubated for 30 min at 37° C., The medium was then replaced with fresh medium containing 30 ng/ml ¹²⁵I-labeled TNF. After 2 hours at 4° C., the cells were lysed with 0.1 N NaOH and the level of bound radioactivity was measured. The level of specific binding of C75R by ¹²⁵I-TNF was significantly decreased after incubation with TRRE. The radioactive count was 1,393 cpm on the cells incubated with TRRE compared to 10,567 cpm on the cells not treated with TRRE, a loss of 87% of binding capacity.

In order to determine the size of the p75 TNF-R cleared from C75R by TRRE, the following experiment was performed. 15×10⁸ C75R cells were seeded in a 150 mm cell culture plate and incubated at 37° C. in 5% CO₂ for 12 to 16 hours. TRRE medium was incubated with C75R cells in the 150 mm plate for 30 min and the resulting supernatant was collected and centrifuged. The concentrated sample was applied to 10% acrylamide SDS-PAGE and electrophoretically transferred to a polyvinylidene difluoride membrane (Immobilon). Immunostaining resulted in a single band of 40 kDa, similar to the size found in biological fluids. Thus, transfected COS-1 cells expressed high levels of human p75 TNF-R in a form similar to native TNF-R.

The following assay method was adopted for routine measurement of TRRE activity. C75R cells and COS-1 cells were seeded into 24-well culture plates at a density of 2.5×10⁵ cells/ml/well and incubated overnight (for 12 to 16 hours) in 5% CO₂ at 37° C. After aspirating the medium in the well, 300 μl of TRRE medium was incubated in each well of both the C75R and COS-1 plates for 30 min in 5% CO₂ at 37° C. (corresponding to A and C mentioned below, respectively). Simultaneously, C75R cells in 24-well plates were also incubated with 300 μl of fresh medium or buffer. The supernatants were collected, centrifuged, and then assayed for the concentration of soluble p75 TNF-R by ELISA.

ELISA assay for released TNF-R (WO 9802140) was performed as follows: Polyclonal antibodies to human p75 TNF-R were generated by immunization of New Zealand white female rabbits (Yamamoto et al. Cell. Immunol. 38:403-416, 1978). The IgG fraction of the immunized rabbit serum was purified using a protein G (Pharmacia Fine Chemicals, Uppsala, Sweden) affinity column (Ey et al. (1978) Immunochemistry 15:429-436, 1978). The IgG fraction was then labeled with horseradish peroxidase (Sigma Chemical Co., St. Louis, Mo.) (Tijssen and Kurstok, Anal. Biochem. 136:451-457, 1984). In the first step of the assay, 5 μg of unlabeled IgG in 100 μl of 0.05 M carbonate buffer (pH 9.6) was bound to a 96-well ELISA microplate (Corning, Corning, N.Y.) by overnight incubation at 4° C. Individual wells were washed three times with 300 μl of 0.2% Tween-20 in phosphate buffered saline (PBS). The 100 μl of samples and recombinant receptor standards were added to each well and incubated at 37° C. for 1 to 2 hours. The wells were then washed in the same manner, 100 μl of horseradish peroxidase-labeled IgG added and incubated for 1 hour at 37° C. The wells were washed once more and the color was developed for 20 minutes (min) at room temperature with the substrates ABTS (Pierce, Rockford, Ill.) and 30% H₂O₂ (Fisher Scientific, Fair Lawn, N.J.). Color development was measured at 405 nm.

When C75R cells were incubated with TRRE medium, soluble p75 TNF-R was released into the supernatant which was measurable by ELISA. The amount of receptors released corresponded to the amount of TRRE added There was also a level of spontaneous TNF-R release in C75R cells incubated with just medium alone. It is hypothesized that this is due to an endogenous source of proteolytic enzyme, a homolog of the human TRRE of monkey origin.

The following calculations were performed. A=(amount of soluble p75 TNF-R in a C75R plate treated with the TRRE containing sample); i.e. the total amount of sTNF-R in a C75R plate. B=(amount of soluble p75 TNF-R spontaneously released in a C75R plate treated with only medium or buffer containing the same reagent as the corresponding samples but without exogenous. TRRE); i.e. the spontaneous release of sTNF-R from C75R cells. C=(amount of soluble p75 TNF-R in a COS-1 plate treated with the TRRE sample or the background level of soluble p75 TNF-R released by THP-1.); i.e. the degraded value of transferred (pre-existing) sTNF-R in the TRRE sample during 30 min incubation in a COS-1 plate. This corresponds to the background level of sTNF-R degraded in a C75R plate. The net release of soluble p75 TNF-R produced only by TRRE activity existing in the initial sample is calculated as follows: (Net release of soluble p75 TNF-R only by TRRE)=A−B−C.

Unit activity of TRRE was defined as follows: 1 pg of soluble p75 TNF-R net release (A-B-C) in the course of the assay is one unit (U) of TRRE activity.

Using this assay, the time course of receptor shedding by TRRE was measured in the following experiment. TRRE-medium was incubated with C75R and COS-1 cells for varying lengths of time. The supernatants were then collected and assayed for the level of soluble p75 TNF-R by ELISA and the net TRRE activity was calculated. Detectable levels of soluble receptor were released by TRRE within 5 min and increased up to 30 min. Longer incubation times showed that the level of TRRE remained relatively constant after 30 min, presumably from the depletion of substrates. Therefore, 30 min was determined to be the optimal incubation time.

The induction patterns of TRRE and known MMPs by PMA stimulation are quite different. In order to induce MMPs, monocytic U-937 cells, fibrosarcoma HT-1080 cells, or peritoneal exudate macrophages (PEM) usually have to be stimulated for one to three days with LPS or PMA. On the other hand, as compared with this prolonged induction, TRRE is released very quickly in culture supernatant following 30 min of PMA-stimulation. The hypothesis that TRRE and sTNF-R form a complex in vitro was confirmed by the experiment that 25% TRRE activity was recovered from soluble p75 TNF-R affinity column. This means that free TRRE has the ability to bind to its catalytic product, sTNF-R. The remaining 75% which did not combine to the affinity column may already be bound to sTNF-R or may not have enough affinity to bind to sTNF-R even though it is in a free form.

### Example 2

### Characterization of TRRE Obtained from THP-1 Cells

TRRE obtained by PHA stimulation of THP-1 cells was partially purified from the culture medium (WO 9802140). First, protein from the medium was concentrated by 100% saturated ammonium sulfate precipitation at 4° C. The precipitate was pelleted by centrifugation at 10,000×g for 30 min and resuspended in PBS in approximately twice the volume of the pellet. This solution was then dialyzed at 4° C. against 10 mM Tris-HCl, 60 mM NaCl, pH 7.0. This sample was loaded on an anion-exchange chromatography, Diethylaminoethyl (DEAE)-Sephadex A-25 column (Pharmacia Biotech) (2.5×10 cm) previously equilibrated with 50 mM Tris-HCl, 60 mM NaCl, pH 8.0. TRRE was then eluted with an ionic strength linear gradient of 60 to 250 mM NaCl, 50 mM Tris-HCl, pH 8.0. Each fraction was measured for absorbance at 280 nm and assayed for TRRE activity. The DEAE fraction with the highest specific activity (the highest value of TRRE units/A280) was pooled and used in the characterizations of TRRE described in this example.

In the next experiment, the substrate specificity of the enzyme was elucidated using immunohistochemical techniques. Fluorescein isothiocyanate (FITC)-conjugated anti-CD54, FITC-conjugated goat anti-rabbit and mouse antibodies, mouse monoclonal anti-CD30, anti-CD11b and anti-IL-1R (Serotec, Washington D.C.) were used. Rabbit polyclonal anti-p55 and p75 TNF-R were obtained according to Yamamoto et al. (1978) Cell Immunol. 38:403-416. THP-1 cells were treated for 30 min with 1,000 and/or 5,000 U/ml of TRRE eluted from the DEAE-Sephadex column, and then transferred to 12×75 mm polystyrene tubes (Fischer Scientific, Pittsburgh, Pa.) at 1×10⁵ cells/100 μl/tube. The cells were then pelleted by centrifugation at 350×g for 5 min at 4° C. and stained directly with 10 μl FITC-conjugated anti-CD54 (diluted in cold PBS/0.5% sodium aside), indirectly with FITC-conjugated anti-mouse antibody after treatment of mouse monoclonal anti-CD11 b, IL-1R and CD30 and also indirectly with FITC-conjugated anti-rabbit antibody after treatment of rabbit polyclonal anti-p55 and p75 TNF-R.

THP-1 cells stained with each of the antibodies without treatment of TRRE were used as negative controls. The tubes were incubated for 45 min at 4° C., agitated every 15 min, washed twice with PBS/2% FCS, repelleted and then resuspended in 200 μl of 1% paraformaldehyde. These labeled THP-1 cells were analyzed using a fluorescence activated cell sorter (FACS) (Becton-Dickinson, San Jose, Calif.) with a 15 mW argon laser with an excitation of 488 nm. Fluorescent signals were gated on the basis of forward and right angle light scattering to eliminate dead cells and aggregates from analysis. Gated signals (10⁴) were detected at 585 BP filter and analyzed using Lysis II software. Values were expressed as percentage of positive cells, which was calculated by dividing mean channel fluorescence intensity (MFI) of stained THP-1 cells treated with TRRE by the MFI of the cells without TRRE treatment (negative control cells).

To test the in vitro TNF cytolytic assay by TRRE treatment the L929 cytolytic assay was performed according to the method described by Gatanaga et al. (1990b). Briefly, L929 cells, an adherent murine fibroblast cell line, were plated (70,000 cells/0.1 ml/well in a 96-well plate) overnight. Monolayered L929 cells were pretreated for 30 min with 100, 500 or 2,500 U/ml of partially-purified TRRE and then exposed to serial dilutions of recombinant human TNF for 1 hour. After washing the plate with RPMI-1640 with 10% FCS to remove the TRRE and TNF, the cells were incubated for 18 hours in RPMI-1640 with 10% FCS containing 1 μg/ml actinomycin D at 37° C. in 5% CO₂. Culture supernatants were then aspirated and 50 μl of 1% crystal violet solution was added to each well. The plates were incubated for 15 min at room temperature. After the plates were washed with tap water and air-dried, the cells stained with crystal violet were lysed by 100 μl per well of 100 mM HCl in methanol. The absorbance at 550 nm was measured using an EAR 400 AT plate reader (SLT-Labinstruments, Salzburg, Austria).

To investigate whether TRRE also truncates the ˜55 kDa size of TNF-R, partially-purified TRRE was applied to THP-1 cells which express low levels of both p55 and p75 TNF-R (approximately 1,500 receptors/cell by Scatchard analysis). TRRE eluate from the DEAE-Sephadex column was added to THP-1 cells (5×10⁶ cells/ml) at a final TRRE concentration of 1,000 U/ml for 30 min. The concentration of soluble p55 and p75 TNF-R in that supernatant was measured by soluble p55 and p75 TNF-R ELISA. TRRE was found to truncate both human p55 and p75 TNF-R on THP-1 cells and released 2,382 and 1,662 pg/ml soluble p55 and p75 TNF-R, respectively.

Therefore, TRRE obtained by PHA stimulation of THP-1 cells is capable of enzymatically cleaving and releasing human p75 TNF-R on C75R cells, and both human p55 and p75 TNF-R on THP-1 cells.

Partial inhibition of TRRE activity was obtained by chelating agents such as 1,10-phenanthroline, EDTA and EGTA (% TRRE activity remaining were 41%, 67% and 73%, respectively, at 2 mM concentration). On the other hand, serine protease inhibitors such as PMSF, AEBSF and 3,4-DCI, and serine and cysteine protease inhibitors such as TLCK and TPCK had no effect on the inhibition of TRRE. TRRE was slightly activated in the presence of Mn²⁺, Ca²⁺, Mg²⁺, and Co²⁺ (% TRRE activities remaining were 157%, 151%, 127%, and 123%, respectively), whereas partial inhibition occurred in the presence of Zn²⁺ and Cu²⁺ (% TRRE activities remaining were 23% and 47%, respectively) (WO 9802140).

TRRE fractions from the most active DEAE fraction (60 mM to 250 mM NaCl) can be purified further. In one method (WO 9802140), the fractions were concentrated to 500 μL with a Centriprep-10 filter (10,000 MW cut-off membrane) (Amicon). This concentrated sample was applied to 6% PAGE under non-denaturing native conditions. The gel was sliced horizontally into 5 mm strips and each was eluted into 1 ml PBS. The eluates were then tested according to the assay (Example 1) for TRRE activity.

### Example 3

### TRRE Activity Alleviates Septic Shock

The following protocol was used to test the effects of TRRE in preventing mortality in a model for septic shock. Mice were injected with lethal or sublethal levels of LPS, and then with a control buffer or TRRE. Samples of peripheral blood were then collected at intervals to establish if TRRE blocked TNF-induced production of other cytokines in the bloodstream. Animals were assessed for the ability of TRRE to block the clinical effects of shock, and then euthanized and tissues examined by histopathological methods.

Details were as follows: adult Balb/c mice, were placed in a restraining device and injected intravenously via the tail vein with a 0.1 ml solution containing 10 ng to 10 mg of LPS in phosphate buffer saline (PBS). These levels of LPS induce mild to lethal levels of shock in this strain of mice. Shock results from changes in vascular permeability, fluid loss, and dehydration, and is often accompanied by symptoms including lethargy, a hunched, stationary position, rumpled fur, cessation of eating, cyanosis, and, in serious cases, death within 12 to 24 hours. Control mice received an injection of PBS. Different amounts (2,000 or 4,000 U) of purified human TRRE were injected IV in a 0.1 ml volume within an hour prior to or after LPS injection. Serum (0.1 ml) was collected with a 27 gauge needle and 1 ml syringe IV from the tail vein at 30, 60 and 90 minutes after LPS injection. This serum was heparinized and stored frozen at −20° C. Samples from multiple experiments were tested by ELISA for the presence of sTNF-R, TNF, IL-8 and IL-6. Animals were monitored over the next 12 hours for the clinical effects of shock. Selected animals were euthanized at periods from 3 to 12 hours after treatment, autopsied and various organs and tissues fixed in formalin, imbedded in paraffin, sectioned and stained by hematoxalin-eosin (H and E). Tissue sections were subjected to histopathologic and immunopathologic examination.

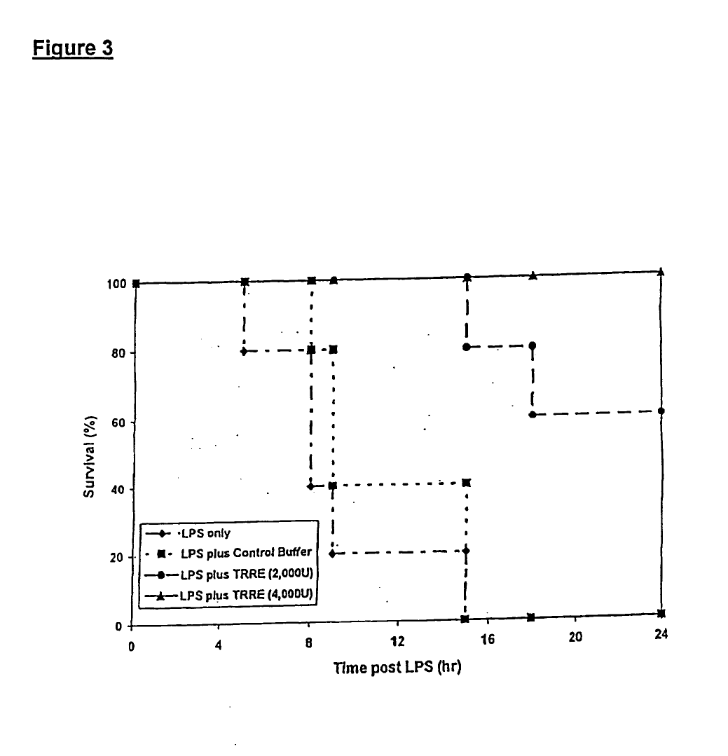

FIG. 3 shows the results obtained. (♦) LPS alone; (▪) LPS plus control buffer; (●) LPS plus TRRE (2,000 U); (▴) LPS plus TRRE (4,000 U).

Mice injected with LPS alone or LPS and a control buffer died shortly after injection. 50% of the test animals were dead after 8 hours (LPS) or 9 hours (LPS plus control buffer), and 100% of the animals were dead at 15 hours. In contrast, animals treated with TRRE obtained as described in Example 1 did much better. When injections of LPS were accompanied by injections of a 2,000 U of TRRE, death was delayed and death rates were lower. Only 40% of the animals were dead at 24 hours. When 4,000 U of TRRE was injected along with LPS, all of the animals had survived at 24 hours. Thus, TRRE is able to counteract the mortality induced by LPS in test-animals.

### Example 4

### TRRE Activity Decreases Tumor Necrotizing Activity

The following protocol was followed to test the effects of TRRE on tumor necrosis in test animals in which tumors were produced, and in which TNF was subsequently injected.

On Day 0, cutaneous Meth A tumors were produced on the abdominal wall of fifteen BALB/c mice by intradermal injection of 2×20⁵ Meth A tumor cells. On Day 7, the mice were divided into three groups of five mice each and treated as follows:
Group 1: Injected intravenously with TNF (1 μg/mouse).
Group 2: Injected intravenously with TNF (1 μg/mouse) and injected intratumorally with TRRE obtained as in Example 1 (400 units/mouse, 6, 12 hours after TNF injection).
Group 3: Injected intravenously with TNF (1 μg/mouse) and injected intratumorally with control medium (6, 12 hours after TNF injection).

On Day 8, tumor necrosis was measured with the following results: Group 1: 100% of necrosis (5/5); Group 2: 20% (1/5); Group 3: 80% (4/5). Injections of TRRE greatly reduced the ability of TNF to induce necrosis in Meth A tumors in BALB/c mice.

Since adding TRRE activity ablates the beneficial necrotizing activity of TNF, blocking endogenous TRRE activity would promote the beneficial effects of TNF.

### Example 5

### Nine New Polynucleotide Clones that Affect TRRE Activity

A number of cells have been found to express high levels of TRRE activity, especially after PMA stimulation. These include the cell lines designated THP-1, U-937, HL-60, ME-180, MRC-5, Raji, K-562. Jurkat cells have a high TRRE activity (850 TRRE U/mL at 10⁻² PMA). In this experiment, the expression library of the Jurkat T cell (ATCC #TIB-152) was obtained and used to obtain 9 polynucleotide clones that augment TRRE activity.

Selection of expression sequences in the library was done by repeated cycles of transfection into COS-1 cells, followed by assaying of the supernatant as in Example 1 for the presence of activity cleaving and releasing the TNF receptor. Standard techniques were used in the genetic manipulation. Briefly, the DNA of 10⁶ Jurkat cells was extracted using an InVitrogen plasmid extraction kit according to manufacturer's directions. cDNA was inserted in the ZAP Express™/EcoRI vector (cat. no. 938201, Stratagene, La Jolla Calif. The library was divided into 48 groups of DNA and transformed into COS-1 cells using the CaCl transfection method. Once the cells were grown out, the TRRE assay was performed, and five positive groups were selected. DNA from each of these five groups was obtained, and transfected into E. coli, with 15 plates per group. DNA was prepared from these cells and then transfected into COS-1 cells once more. The cells were grown out, and TRRE activity was tested again. Two positive groups were selected and transfected into E. coli, yielding 98 colonies. DNA was prepared from 96 of these colonies and transfected into COS-1 cells. The TRRE activity was performed again, and nine clones were found to substantially increase TRRE activity in the assay. These clones were designated 2-8,2-9, 2-14, 2-15, P2-2, P2-10, P2-13, P2-14, and P2-15.

FIG. 4 is a bar graph showing the TRRE activity observed when the 9 clones were tested with C75 cells in the standard assay (Example 1).

These nine clones were then sequenced according to the following procedure:
1. Plasmid DNA was prepared using a modified alkaline lysis procedure.
2. DNA sequencing was performed using DyeDeoxy termination reactions (ABI). Base-specific fluorescent dyes were used as labels.
3. Sequencing reactions were analyzed on 5.75% Long Ranger™ gels by an ABI 373A-S or on 5.0% Long Ranger™ gels by an ABI 377 automated sequencer.
4. Subsequent data analysis was performed using Sequencher™ 3.0 software.
   Standard primers T7X, T3X, -40, -48 Reverse, and BK Reverse (BKR) were used in sequencing reactions. For each clone, several additional internal sequencing primers (listed below) were synthesized.

NCBI BLAST (Basic Local Alignment Search Tool) sequence analysis (Altschul et al. (1990) J. Mol. Biol. 215:403-410) was performed to determine if other sequences were significantly similar to these sequences. Both the DNA sequences of the clones and the corresponding ORFs (if any) were compared to sequences available in databases.

The following clones were obtained and sequenced:

TABLE 1
DNA sequences affecting TRRE activity

| | | | | | Related |
|---|---|---|---|---|---|
| | | SEQ | Approx | | Sequences |
| | Sequence | ID | Length | Expression | (potential |
| Clone | Designation | NO: | (bp) | Designation | homology) |
| 2-9 | AIM2 | 1 | 4,047 | | — |
| 2-8 | AIM3T3 | 2 | 739 | | M. musculus |
| | (partial | | | | 45S pre-rRNA |
| | sequence) | | | | gene |
| | AIM3T7 | 3 | 233 | | |
| | (partial | | | | |
| | sequence) | | | | |
| 2-14 | AIM4 | 4 | 2,998 | Mey3 | human arfaptin |
| | | | | | 2 and others |
| | | | | | (see below) |
| 2-15 | AIM5 | 5 | 4,152 | | — |
| P2-2 | AIM6 | 6 | 3,117 | Mey5 | — |
| P2-10 | AIM7 | 7 | 3,306 | Mey6 | Human Insulin- |
| | | | | | like Growth |
| | | | | | factor II |
| | | | | | Receptor |
| P1-13 | AIM8 | 8 | 4,218 | | — |
| P2-14 | AIM9 | 9 | 1,187 | Mey8 | — |
| P2-15 | AIM10 | 10 | 3,306 | | E1b-55 kDa- |
| | | | | | associated |
| | | | | | protein |

Clone 2-9 (AIM2): The internal primers used for sequencing are shown in SEQ. ID NOS:11-38. The sequence of AIM2 is presented in SEQ ID NO:1. The complementary strand of the AIM2 sequence is SEQ ID NO:147. The longest open reading frame (ORF) in the AIM2 sequence is 474 AA long and represented in SEQ ID NO:148.

Clone 2-8 (AIM3): Two partial sequences of length 739 and 233 were obtained and designated AIM3T3 and AIM3T7. The internal primers used for sequencing are shown in SEQ. ID NOS:3946. The sequences of AIM3T3 and AIM3T7 are presented in SEQ ID NOs:2 and 3, respectively. The BLAST search revealed that the AIM3T3 sequence may be homologous to the mouse (M. musculus) 28S ribosomal RNA (Hassouna et al. Nucleic Acids Res. 12:3563-3583, 1984) and the M. musculus 45S pre-rRNA genes (Accession No. X82564. The complementary sequence of the AIM3T3 sequence showed 99% similarity over 408 bp beginning with nt 221 of SEQ ID NO:2 to the former and 97% similarity over the same span to the latter.

Clone 2-14 (AIM4). The internal primers used for sequencing are shown in SEQ. ID NOS:14-65. The sequence of AIM4 is presented in SEQ ID NO:4. The complementary strand of the AIM4 sequence is SEQ ID NO:149. The longest ORF in the AIM4 sequence is 236 M long and represented in SEQ ID NO:150. AIM4 has significant alignments to human sequences arfaptin 2, ADE2H1 mRNA showing homologies to SAICAR synthetase, polypyrimidine tract binding protein (heterogeneous nuclear ribonucleoprotein I) mRNA, several PTB genes for polypirimidine tract binding proteins, mRNA for por1 protein. Human arfaptin 2 is a putative target protein of ADP-ribosylation factor that interacts with RAC1 by binding directly to it. RAC1 is involved in membrane ruffling. Arfaptin 2 has possible transmembrane segments, potential CK2 phosphorylation sites, PKC phosphorylation site and RGD cell attachment sequence.

Clone 2-15 (AIM5): The internal primers used for sequencing are shown in SEQ. ID NOS:66-80. The sequence of AIM5 is presented in SEQ ID NO:5. The BLAST search revealed that the AIM5 sequence displays some similarity to Human Initiation Factor 5A (eIF-5A) Koettnitz et al. (1995) Gene 159:283-284, 1995 and Human Initiation Factor 4D (eIF 4D) Smit-McBride et al. (1989) J. Biol. Chem. 264:1578-1583, 1989.

Clone P2-2 (AIM6): The internal primers used for sequencing are shown in SEQ. ID NOS:81-93. The sequence of AIM6 is presented in SEQ ID NO:6. The longest ORF in the AIM6 sequence is 1038 M long and represented in SEQ ID NO:151.

Clone P2-10 (AIM7): The internal primers used for sequencing are shown in SEQ. ID NOS:94-106. The sequence of AIM7 is presented as SEQ ID NO:7. The longest ORF in the AIM7 sequence is 849 M long and represented in SEQ ID NO:152. The BLAST search revealed that this clone may be related to the Human Insulin-like Growth Factor II Receptor (Morgan et al. Nature 329:301-307, 1987 or the Human Cation-Independent Mannose 6-Phosphate Receptor mRNA (Oshima et al. J. Biol. Chem. 263:2553-2562, 1988). The AIM7 sequence showed roughly 99% identity to both sequences over 2520 nucleotides beginning with nt 12 of SEQ ID NO:7 and 99% similarity to the latter over the same span.

Clone P2-13 (AIM8): The internal primers used for sequencing are shown in SEQ. ID NOS:107-118. The sequence of AIM8 is presented as SEQ ID NO:8. The longest ORF in the AIM8 sequence is 852 AA long and represented in SEQ ID NO:153.

Clone P2-14 (AIM9): The internal primers used for sequencing are shown in SEQ. ID NOS:119-124. The sequence of AIM9 is presented as SEQ ID NO:9. The longest ORF was about 149 amino acids in length.

Clone P2-15 (AIM10): The internal primers used for sequencing are shown in SEQ. ID NOS:125-146. The sequence of AIM10 is presented as SEQ ID NO:10. The longest ORF in the AIM10 sequence is 693 AA long and represented in SEQ ID NO:154. Sequence 10 on BLASTN search of non-redundant databases at NCBI aligns with Human mRNA for E1b-55 kDa-associated protein, locus HSA7509 (Accession AJ007509, NID g3319955).

Clonal DNA may be directly injected into test animals in order to test the ability of these nucleic acids to induce TRRE activity, counteract septic shock and/or affect tumor necrosis, as is described in detail in Examples 3 and 4. Alternatively, proteins or RNA can be generated from the clonal DNA for similar testing.

### Example 6

### Expression of Newly Obtained Clones

Example 5 describes 9 new clones which enhance TRRE activity in a cell surface assay system. The clones were obtained in the pBK-CMB Phagmid vector.

The following work was done on contract through the commercial laboratory Lark Technologies, Houston, Tex. The clones were removed from shuttle vectors and inserted into expression vectors in the following manner. Recombinant plasmid (pBK-CMV containing insert) was digested with appropriate restriction enzyme(s) such as Spe I, Xba I, EcoR I or others, as appropriate. The Baculovirus Transfer Vector (pAcGHLT-A Baculovirus Transfer Vector, PharMingen, San Diego, Calif., Cat. No. 21460P) was also cut with appropriate restriction enzyme(s) within or near the multiple cloning site to receive the insert removed from the shuttle vector.

The fragment of interest being sublconed was isolated from the digest using Low-Melting agarose electrophoresis and purified from the gel using a Qiaquick Gel Extraction Kit following Lark SOP MB 020602. If necessary, the receiving vector was treated with alkaline phosphatase according to Lark SOP MB 090201. The fragment was ligated into the chosen site of the vector pAcGHLT-A. The recombinant plasmid was transformed into E coli XL1 Blue MRF′ cells and the transformed bacterial cells were selected on LB agar plates containing ampicillin (100 μg/ml). Ampicillin resistant colonies were picked and grown on LB broth containing ampicillin for plasmid preparation.

Plasmid DNA was prepared using Alkaline Minilysate Procedure (Lark SOP MB 010802 and digested with appropriate restriction enzyme(s). Selected subclones were confirmed to be of the correct size. Sublcones were digested with other appropriate restriction enzyme(s) to ascertain correct orientation of the insert by confirming presence of fragments of proper size(s). A subclone was grown in 100 ml of LB broth containing ampicillin (100 μg/ml) and the plasmid DNA prepared using Qiagen Midi Plasmid Preparation Kit (Lark SOP MB 011001). The DNA concentration was determined by measuring the absorbance at 260 nm and the DNA sample was verified to be originated from correct subclone by restriction digestion.

Thus were produced the expression constructs for Mey3, Mey5, Mey6, Mey8 now with the coding sequence of interest fused to GST gene with polyhistitidine tag, protein kinase A site and thrombin cleavage site. The GST gene and now the fusion protein are under the polyhedrin promotor. PharMingen (San Diego, Calif.) incorporated the vector with insert into functional baculovirus particles by co-inserting the transfer vector (pAcGHLT) into susceptible insect cell line S along with linearized virus DNA (PharMingen, San Diego, Calif., BaculoGold viral DNA, Cat. No. 21100D). The functional virus particles were grown again on the insect cells to generate a high titer stock. Protein production was then done by infecting a large culture of cells in Tini cell. The cells were harvested when the protein yield reached a maximum and before the virus killed the cells. Fusion proteins were collected on a glutatione-agarose column, washed and released with glutathionine.

Proteins collected from the affinity column were quantified by measuring OD₂₈₀ and were assayed on gels using SDS-PAGE and Western blotting with labeled anti-GST (PharMingen, San Diego, Calif., mAbGST Cat. No. 21441A) to confirm that all the bands present included the GST portion.

Four of the ten sequences have been cloned, expressed in bacculovirus infected insect cells, and then purified.

TABLE 2
Expressed protein from Jurkat library clones

| | | Amount of protein |
|---|---|---|
| Name | Sequence in insert | (mg/mL) |
| Mey3 | AIM4 | 4.7, 5.0 |
| Mey5 | AIM6 | 1.36, 1.50 |
| Mey6 | AIM7 | 0.33 |
| Mey8 | AIM9 | 1.53 |

Gels indicated the presence of the GST protein in addition to larger proteins that were also positive with the anti-GST antibody in Western analyses. Mey3 repeatedly exhibited the presence of proteins around 32 kDa, 56 kDa, bands around 60-70 kDa and another larger than 70 kDa. Mey5 consistently had proteins migrating as approximately 34 kDa, 38 kDa, 58 kDa, around 60-70 kDa, and others larger than 70 kDa. Mey6 had protein bands around 34 kDa, 56 kDa, 58 kDa, and bands around 60-70 kDa. Mey8 had protein bands around 36 kDa, 58 kDa and bands around 60-70 kDa. All of the indicated bands were positive for GST. The bands may represent the desired fusion protein or degradation/cleavage product generated during growth and purification.

### Example 7

### Assay of Expression Products for Effect on TNF-R Cleaving Activity

The following method was used to measure TRRE activity of Mey 3, 5, 6 and 8. C75R cells and COS-1 cells were seeded into 24-well culture plates at a density of 2.5×10⁵ cells/ml/well and incubated overnight (for 12 to 16 hours) in 5% CO₂ at 37° C. After aspirating the medium in the well, 300 μl of 1 ug of Mey 3, 5 and 8 were incubated in each well of both the C75R and COS-1 plates for 30 min in 5% CO₂ at 37° C. (corresponding to A and C mentioned below, respectively). Simultaneously, C75R cells in 24-well plates were also incubated with 300 μL of fresh medium or buffer (corresponding to B mentioned below). The supernatants were collected, centrifuged, and then assayed for the concentration of soluble p75 TNF-R by ELISA as described in Example 1.

The following results were obtained:

TABLE 3
Enzymatic activity of expressed clones

| | TNF-receptor releasing activity |
|---|---|
| Clone No. | U/mg |
| Mey-3 | 341 |
| Mey-5 | 671 |
| Mey-6 | 452 |
| Mey-8 | 191 |

### Example 8

### Effectiveness of Expression Products in Treating Septic Shock

The protocol outlined in Example 3 was used to test the effects of the expression products from the new clones in preventing mortality in the septic shock model.

Different amounts of recombinant Mey 3, 5, and 8 (10-100 ug/mouse) were injected i.v. in a 0.05 ml volume within an hour prior to or after injection of a lethal dose of LPS. Serum (0.1 ml) was collected using a 27 gauge needle and 1 ml syringe from the tail vein at 30, 60 and 90 minutes after LPS injection. This serum was heparinized and stored frozen at −20° C. Samples from multiple experiments were tested by ELISA for the presence of solubilized TNR-R, the TNR ligand, IL-8, and IL-6. Animals were monitored over the next 12 hours for the clinical effects of shock. Selected animals were euthanized from 3 to 12 hours after treatment, autopsied and various organs and tissues fixed in formalin, imbedded in paraffin, sectioned and stained by hematoxalin-eosin (H and E). Tissue sections were subjected to histopathologic and immunopathologic examination.

FIG. 5 shows the results obtained. (♦) saline; (▪) BSA; (Δ) Mey-3 (100 μg); (X) Mey-3 (10 μg); (*) Mey-5 (10 μg); (●) Mey-8 (10 μg).

Mice injected with LPS alone or LPS, a control buffer or control protein (BSA) died rapidly. All of the animals in this group were dead at 24 hours. In contrast, when injections of LPS were accompanied by injections of a 10-100 ug of Mey 3, 5 and 8, death was delayed and death rates were lower. None of the animal were dead at 24 hours that had been treated with Mey 3 and Mey 5. Only 66% of the animals were dead at 24 hours that had been treated with Mey 8. Thus, Mey 3, 5 and 8 were able to counteract the mortality induced by LPS in test animals.

1-32. (canceled)

33. A method for treating arthritis or reducing inflammation in a subject, comprising administering to the subject a means for cleaving TNF receptors from the surface of cells.

34. The method of claim 33, wherein the receptor cleaving means is a protein containing an amino acid sequence encoded in any of SEQ. ID NOs:1-10, or fragment of any of said amino acid sequences that causes release of TNF receptor from human cells in which TNF is expressed.

35. The method of claim 33, wherein the receptor cleaving means is a protein containing a sequence that is at least 90% identical to an amino acid sequence encoded in SEQ. ID NO:8, or fragment thereof that causes increased release of TNF receptor from human cells in which TNF is expressed.

36. The method of claim 33, wherein the receptor cleaving means is a protein containing an amino acid sequence encoded in SEQ. ID NO:8, or fragment thereof that causes increased release of TNF receptor from human cells in which TNF is expressed.

37. The method of claim 33, wherein the receptor cleaving means is an isolated naturally occurring protein obtainable by a process comprising:
a) stimulating THP-1 cells with phorbol myristate acetate;
b) harvesting culture medium from the stimulated cells; and
c) isolating from the medium a protein that causes TNF receptor cleavage.

38. The method of claim 37, wherein the protein has been isolated by a method comprising ion exchange chromatography and separation by molecular weight.

39. The method of claim 37, wherein the protein has been obtained from THP-1, U-937, HL-60, ME-180, MRC-5, Raji, or K-562 cells or normal human monocytes.

40. The method of claim 33, wherein the TNF receptors are human p75 TNF receptors.

41. The method of claim 33, wherein the TNF receptors are human p55 TNF receptors.

42. The method of claim 33, wherein the receptor cleaving means is a protein that is at least about 90% homogeneous when analyzed by SDS polyacrylamide gel electrophoresis.

43. The method of claim 33, comprising administering the protein cleaving means systemically.

44. The method of claim 33, comprising administering the protein cleaving means near the inflammation.

45. The method of claim 34, comprising administering the protein cleaving means intravenously.

46. The method of claim 34, comprising administering the protein cleaving means intramuscularly.

47. The method of claim 33, whereby the subject is treated for sepsis.

48. The method of claim 33, whereby the subject is treated for arthritis.

49. The method of claim 33, whereby the subject is treated for rheumatoid arthritis.

50. The method of claim 33, whereby the subject is treated for multiple sclerosis.
